(19) United States
(12) Patent Application Publication
(10) Pub. No.: US 2007/0178462 A1
(43) Pub. Date: Aug. 2, 2007

(54) Nucleic acid molecules encoding novel murine low-voltage activated calcium channel proteins, designated-alpha1h, encoded proteins and methods of use thereof

(76) Inventors: Victor N. Uebele, Wyncote, PA (US); Thomas M. Connolly, Lansdale, PA (US)

Correspondence Address: MERCK AND CO., INC, P O BOX 2000, RAHWAY, NJ, 07065-0907, US

(21) Appl. No.: 10/589,702

(22) PCT Filed: Feb. 14, 2005
(86) PCT No.: PCT/US05/04432

§ 371 Date: Aug. 17, 2006

Related U.S. Application Data
(60) Provisional application No. 60/545,446, filed on Feb. 18, 2004.

(51) Int. Cl. C12Q 1/68; G01N 33/567; C07H 21/04; C12P 21/06; C07K 14/705; C07K 16/28; C12N 5/06

(52) U.S. Cl. 435/6; 435/7.2; 435/69.1; 435/320.1; 435/353; 530/350; 536/23.5; 530/388.22

(57) ABSTRACT

Disclosed herein are novel nucleic acid molecules encoding murine low-voltage activated calcium channel proteins, designated-α1H, encoded proteins, vectors, host cells transformed therewith, as well as pharmaceutical compositions. Methods of using any of the foregoing, e.g., methods for screening for candidate agonists or antagonists utilizing the novel protein isoforms are also disclosed.

### BACKGROUND OF THE INVENTION

The present invention relates to novel nucleic acid molecules, encoded proteins, vectors, host cells transformed therewith, antibodies reactive with said proteins, as well as pharmaceutical compositions. Methods of using any of the foregoing, e.g., methods for screening for candidate agonists or antagonists utilizing the novel protein isoforms are also contemplated by the present invention.

Calcium is an essential signaling molecule for many normal physiological functions in the human body. These include all electrical signaling in the nervous system, as well as controlling heart and smooth muscle contraction, and hormone release. The entry of calcium into cells is regulated by a diverse set of proteins called calcium channels.

Calcium channels were discovered in 1958 by Fatt and Ginsborg when they explored the ionic basis of a Na+-independent action potential in crab muscle. The most unique and crucial role of Ca²⁺ channels is to translate the electrical signal on the surface membrane into a chemical signal within the cytoplasm, which, in general, increases the intracellular second messenger Ca²⁺, which, in turn, activates many crucial intracellular processes including contraction, secretion, neurotransmission and regulation of enzymatic activities and gene expression. Tsien et al., (1988), Trends Neurosci., vol. 11, pp. 431-438. As might be expected from their central role in signal transduction, Ca²⁺ channels are tightly regulated by a range of signal transduction pathways in addition to regulation by their intrinsic, voltage-dependent gating processes.

Continuing studies have revealed that there are multiple types of Ca²⁺ currents as defined by physiological and pharmacological criteria. See, e.g., Catterall, W. A., (2000) Annu. Rev. Cell Dev. Biol., 16:521-55; Llinas et al, (1992) Trends Neurosci, 15;351-55; Hess, P. (1990) Ann. Rev. Neurosci. 56:337; Bean, B. P. (1989) Ann. Rev. Physiol. 51:367-384; and Tsien et al. (1988) Trends Neurosci. 11:431-38. In addition to exhibiting distinct kinetic properties, different Ca²⁺ channel types can be localized on different regions of a cell with complex morphology. Finally, Ca²⁺ channels in different tissues display different pharmacological profiles, suggesting the possibility of drugs selective for particular organs.

The calcium in nerve cells plays an important role in delivering signals between nerve cells. Calcium has many different delivery paths, however, when delivering peripheral stimuli, the voltage-activated calcium channel is crucial. Voltage activated channels play important roles including neuroexcitation, neurotransmitter and hormone secretion, and regulation of gene transcription through Ca-dependent transcription factors. Their functions depend in part on their cellular localization and their gating properties (characteristics of their opening, inactivation, deactivation, and recovery from inactivation). Five general classes of voltage activated calcium channels have been observed in various neuronal and non-neuronal tissues. The complement of calcium subunits and the subcellular localization of the expressed voltage activated calcium channels determine the functional cellular properties.

Native calcium channels have been classified by their electrophysiological and pharmacological properties as T, L, N, P and Q types (for views see McCleskey, E. W. et al. Curr Topics Membr (1991) 39:295-326, and Dunlap, K. et al. Trends Neurosci (1995) 18:89-98). Voltage-gated calcium channels can be divided into Low Voltage Activated calcium channel (LVA) that is activated at a lower voltage and High Voltage Activated (HVA) calcium channel that is activated at a higher voltage than the resting membrane potential. HVA channels are currently known to comprise at least three groups of channels, known as L-, N- and P/Q-type channels. These channels have been distinguished from one another electrophysiologically as well as biochemically on the basis of their pharmacology and ligand binding properties. The L, N, P and Q-type channels activate at more positive potentials (high voltage activated) and display diverse kinetics and voltage-dependent properties. A fourth type of high voltage-activated calcium channel (Q-type) has been described, although whether the Q- and P-type channels are distinct molecular entities is controversial (Sather, W. A et al. Neuron (1995) 11:291-303; Stea, A. et al. Proc Natl Acad Sci USA (1994) 91:10576-10580; Bourinet, E. et al. Nature Neuroscience (1999) 2:407415).

To date, only one type of low-threshold calcium channel is known, the T-type calcium channel. These channels are so called because they carry a transient current with a low voltage of activation and rapid inactivation. (Ertel and Ertel (1997) Trends Pharmacol. Sci. 18:37-42.) In general, T-type calcium channels are involved in the generation of low threshold spikes to produce burst firing (Huguenard, 1996). The main factor which defines the different calcium currents is which α₁ subtype is included in the channel complex. The subfamily of α_{1G}, α_{1H} and α_{1I} subunits display the low-voltage activation characteristic of T-type channels.

One low-T type and five high VGCC types (L, N, P, Q, R) have been studied through pharmacological and electrophysiological studies. Three genes have been identified for the α₁ subunits of LVA channels, reviewed in Hofmann et al., (1999), Rev. Physiol. Biochem. Phamacol. 139:33-87; Lacinova et al.,(2000) Gen. Physiol. Biophys., 19: 121-36).

Although only the pore-forming subunits of three members of T-type calcium channels have been cloned until now (Perez-Reyes, 1998; Perez-Reyes et al., 1998; Lee et al., 1999; Lacinova et al., 2000; Lory et al., 2000; McRory et al., 2001), the L-type subfamily has been characterized extensively by biochemical approaches. These studies have revealed that the L-type calcium channel complex is a heteropentamer consisting of α1, β, α/δ and γ subunits, The predicted structure of the α₁ subunit consists of four repeating motifs (MI-MIV), each motif comprising six hydrophobic segments (S1-S6). A highly conserved segment connecting the S5 and S6 transmembrane domains in each motif, termed the P loop or ‘SS1-SS2’ region, is responsible for calcium selectivity in the pore region (FIG. 1B) (Catterall, 1988; Varadi et al., 1999).

For calcium channels to be effective, Ca²⁺ ions must enter selectively through the pore of the α₁ subunit, bypassing competition with other extracellular ions (Catterall, 1988; Imoto, 1993; Varadi et al., 1995, 1999; Randall and Benham, 1999). The molecular “pores” that flood the surface of voltage gated calcium channels “open” in response to the depolarization of the membrane voltage, which allows for the selective influx of Ca²⁺ ions from an extracellular environment into the interior of a cell. The “opening” of the pores essentially requires a depolarization to a certain level of the potential difference between the inside of the cell bearing the channel and the extracellular medium bathing the cell. The rate of influx of Ca²⁺ into the cell depends on this potential difference. When the accumulating Ca²⁺ reaches a sufficient concentration, it can activate ion channels such as Ca²⁺ activated K⁺ channels that allow positive charge out the cell and thereby repolarize the membrane. It can be seen how calcium channels serve as elements that can sense, amplify, and terminate electrical signals.

T-type channels are located in cardiac & vascular smooth muscle; and in the nervous system. Perez-Reyes et al. discuss the molecular characterization of a neuronal low-voltage-activated T-type calcium channel (Nature 391, 896-900, 1998). Generally, T-type channels are thought to be involved in pacemaker activity, low-threshold calcium spikes, neuronal oscillations and resonance, and rebound burst firing. See F. R. Buhler, J. Hypertension supplement 15(5):s3-7, 1997; B. Cremers et al., J. Cardiovascular Pharmacology, vol. 29(5), pp. 692-6, 1997. The functional roles for T-type calcium channels in neurons include, inter alia, membrane depolarization, calcium entry and burst firing. (White et al. (1989) Proc. Natl. Acad. Sci. USA 86:6802-6806.) The LVA channels differ from HVA channels in a number of ways, i.e., length of I-II intracellular linker etc and the β subunit does not appear to be associated with α₁ in the LVA class. As well, they lack the canonical sequence that is known to be crucial for beta subunit binding. See Lambert et al., J. Neurosci., 17; 6621-6628, 1997; Leuranguer et al., Neuropharmacology, 37: 701-708, 1998.

Functionally unique Ca channels allow for temporal and spatial control of intracellular calcium ([Ca]ᵢ) and support regulation of cellular activity. T-type calcium channels have more negative activation ranges and inactivate more rapidly than other calcium channels. When the range of membrane potentials for activation and inactivation overlap, these channels can undergo rapid cycling between open, inactivated, and closed states, giving rise to continuous calcium influx in a range of negative membrane potentials where HVA channels are not normally activated. The membrane depolarizing influence of T-type calcium channel activation can become regenerative and produce calcium action potentials and oscillations.

Increases in [Ca]ᵢ, occurring in part via activation of voltage-dependent T-type calcium channels, are important for the orderly progression of the cell cycle and may contribute to the regulation of cell proliferation and growth (Berridge et al. 1998; Ciapa et al. 1994; Guo et al. 1998. Alterations in the density of T-type calcium channel currents and oscillations in [Ca]ᵢ have been described in a variety of organisms (Day et al. 1998; Kono et al. 1996; Kuga et al. 1996; Mitani 1985).

In addition to the variety of normal physiological functions mediated by calcium channels, they are also implicated in a number of human disorders. For example, changes to calcium influx into neuronal cells may be implicated in conditions such as epilepsy, stroke, brain trauma, Alzheimer's disease, multiinfarct dementia, other classes of dementia, Korsakoff's disease, neuropathy caused by a viral infection of the brain or spinal cord (e.g., human immunodeficiency viruses, etc.), amyotrophic lateral sclerosis, convulsions, seizures, Huntington's disease, amnesia, pain transmission, cardiac pacemaker activity or damage to the nervous system resulting from reduced oxygen supply, poison or other toxic substances (See e.g., Goldin et al., U.S. Pat. No. 5,312,928). Other pathological conditions associated with elevated intracellular free calcium levels include muscular dystrophy and hypertension (Steinhardt et al., U.S. Pat. No. 5,559,004).

Recently, mutations identified in human and mouse calcium channel genes have been found to account for several disorders including, familial hemiplegic migraine, episodic ataxia type 2, cerebellar ataxia, absence epilepsy and seizures. Fletcher, et al. (1996) “Absence epilepsy in tottering mutant mice is associated with calcium channel defects.” Cell 87:607-617; Burgess, et al. (1997) “Mutation of the Ca²⁺ channel P subunit gene Cchb4 is associated with ataxia and seizures in the lethargic (1h) mouse.” Cell 88:385-392; Ophoff, et al. (1996) “Familial hemiplegic migraine and episodic ataxia type-2 are caused by mutations in the Ca²⁺ channel gene CACNL1A4.” Cell 87:543-552; Zhuchenko, O. et al. (1997) “Autosomal dominant cerebellar ataxia (SCA6) associated with the small polyglutamine expansions in the UIA-Voltage-dependent calcium channel.” Nature Genetics 15:62-69. The clinical treatment of some disorders has been aided by the development of therapeutic calcium channel antagonists. Janis, et al. (1991) in Calcium Channels: Their Properties, Functions, Regulation and Clinical Relevance. CRC Press, London.

Significantly, changes to calcium influx into cardiovascular cells are implicated in conditions such as cardiac arrhythmia, angina pectoris, hypoxic damage to the cardiovascular system, ischemic damage to the cardiovascular system, myocardial infarction, and congestive heart failure (Goldin et al., supra). More, T-type calcium channels have been implicated in cellular growth and proliferation, particularly in the cardiovascular system (Katz, A. M, Eur. Heart J. Suppl., H18-H23, 999; Lijnen and Petrov, Exp. Clin. Pharmacol., 21: 253-259, 1999; Richard and Nargeot, Electrophsiol. Meet., 123-132, 1998; Wang et al., Am. J. Physiol. 265: C1239-C1246, 1993. Of equal import is the observation that there is limited knowledge in the art of the role of calcium channel types in cell growth control and abnormalities of calcium channels leading to cancer development.

The low threshold spikes and rebound burst firing characteristic of T-type calcium currents is prominent in neurons from inferior olive, thalamus, hippocampus, lateral habenular cells, dorsal horn neurons, sensory neurons (DRG, no dose), cholinergic forebrain neurons, hippocampal intraneurons, CA1, CA3 dentate gyrus pyramidal cells, basal forebrain neurons, amygdaloid neurons (Talley et al., J. Neurosci., 19: 1895-1911, 1999) and neurons in the thalamus. (Suzuki and Rogawski , Proc. Natl. Acad. Sci. USA 86:7228-7232, 1998). As well, T-type channels are prominent in the soma and dendrites of neurons that reveal robust Ca-dependent burst firing behaviors such as the thalramic relay neurons and cerebellar Purkinje cells (Huguenard, J. R., Annu. Rev. Physiol., 329-348, 1996. Consequently, improper functioning of these LVA channels has been implicated in arrhythmias, chronic peripheral pain, improper pain transmission in the central nervous system to name a few.

For example, the data show that T-type channels promote oscillatory behavior which has important consequences for epilepsy. The ability of a cell to fire low threshold spikes is critical in the genesis of oscillatory behavior and increased burst firing (groups of action potentials separated by about 50-100 ms). T-type calcium channels are believed to play a vital role in absence epilepsy, a type of generalized non-convulsive seizure. The evidence that voltage-gated calcium currents contribute to the epileptogenic discharge, including seizure maintenance and propagation includes 1) a specific enhancement of T-type currents in the reticular thalamic (nRT) neurons which are hypothesized to be involved in the genesis of epileptic seizures in a rat genetic model (GAERS) for absence epilepsy (Tsakiridou et al., J. Neurosci., 15: 3110-3117, 1995); 2) antiepileptics against absence petit mal epilepsy (ethosuximide and dimethadione) have been shown at physiologically relevant doses to partially depress T-type currents in thalamic (ventrobasal complex) neurons (Coulter et al., Ann. Neurol., 25:582-93, 1989; U.S. Pat. No. 6,358,706 and references cited therein); and 3) T-type calcium channels underlie the intrinsic bursting properties of particular neurons that are hypothesized to be involved in epilepsy (nRT, thalamic relay and hippocampal pyramidal cells) (Huguenard, supra). The rat α_{1G} is highly expressed in thalamocortical relay cells (TCs) which are capable of generating prominent Ca²⁺-dependent low-threshold spikes (Talley et al., J. Neurosci., 19: 1895-1911, 1999).

The T-type calcium channels have also been implicated in thalamic oscillations and cortical synchrony, and their involvement has been directly implicated in the generation of cortical spike waves that are thought to underlie absence epilepsy and the onset of sleep (McCormick and Bal, Annu. Rev. Neurosci., 20: 185-215, 1997). Oscillations of neural networks are critical in normal brain function such during sleep-wave cycles. It is widely recognized that the thalamus is intimately involved in cortical rhythmogenesis. Thalamic neurons most frequently exhibit tonic firing (regularly spaced spontaneous firing) in awake animals, whereas phasic burst firing is typical of slow-wave sleep and may account for the accompanying spindling in the cortical EEG. The shift to burst firing occurs as a result of activation of a low threshold Ca²⁺ spike which is stimulated by synaptically mediated inhibition (i.e., activated upon hyperpolarization of the RP). The reciprocal connections between pyramidal neurons in deeper layers of the neocortex, cortical relay neurons in the thalamus, and their respective inhibitory interneurons are believed to form the elementary pacemaking circuit. That anti-epileptic drugs cause a reduction of the low-threshold calcium current (LTCC or T-type Ca²⁺ current) in thalamic neurons is evident from the prior art. See Coulter et al.(1989) Ann. Neurol. 25:582-593.) For example, ethosuximide, an anti-epileptic drug has been shown to fully block T-type Ca²⁺ current in freshly dissected neurons from dorsal root ganglia (DRG neurons) of adult rats (Todorovic and Lingle, J. Neurophysiol. 79:240-252, 1998), and may have limited efficacy in the treatment of abnormal, chronic pain syndromes that follow peripheral nerve damage.

T-type channels have also been implicated in contributing to spontaneous fluctuations in intracellular calcium concentrations [Ca]i. Changes to calcium influx into cardiovascular cells, in turn, may be implicated in conditions such as cardiac arrhythmia, angina pectoris, hypoxic damage to the cardiovascular system, ischemic damage to the cardiovascular system, myocardial infarction, and congestive heart failure (Goldin et al., supra).

Other pathological disease states associated with dysfunctional calcium channels, e.g., elevated intracellular free calcium levels include muscular dystrophy and hypertension (Steinhardt et al., U.S. Pat. No. 5,559,004). Consequently, T-type calcium channels are important in pacemaker activity and therefore heart rate in the heart, and in vesicle release from non-excitable cells (Ertel et al.. In cardiovasc. Drugs Ther., 723-739, 1997). It is believed that therapeutic moieties capable of blocking the T-type channel in specific conformational states will find use in the treatment of tachycardia (by decreasing the heart rate) while having little effect on the inotropic properties of the normal heart. See Rousseau et al., J.Am. Coll. Cardiol., 28: 972-979, 1996. According to Sen and Smith, Circ. Res., 75: 149-55, 1994, in a particular cardiomyopathic disease (genetic Syrian hamster model), the disease status results from calcium overload due to an increased expression of T-type calcium channels in ventricular myocytes.

Likewise, researchers have shown that there are increased T-type currents in atrial myocytes from adult rats with growth hormone-secreting tumors. See also Xu and Best, Proc. Natl. Acad. Sci. U.S.A., 87: 4655-4659, 1990; U.S. Pat. No. 6,358,706 and references cited therein. Consequently, a specific T-type calcium channel blocker would find use as a cardioprotectant in these cases.

It is well documented that cortisol is the precursor for glucocorticoids and prolonged exposure to glucocorticoids causes breakdown of peripheral tissue protein, increased glucose production by the liver and mobilization of lipid from the fat depots. Furthermore, individuals suffering from anxiety and stress produce abnormally high levels of glucocorticoids. Consequently, drugs that would regulate these levels would aid in the treatment of stress disorders, e.g., antagonists to CRF. In this regard, the observations of Enyeart et al., Mol. Endocrinol., 7:1031-1040, 1993, that T-type channels in adrenal zona fasciculata cells of the adrenal cortex modulate cortisol secretion will greatly aid in the identification of such a therapeutic candidate.

T-type calcium channels may also be involved in release of nutrients from testis Sertoli cells. Sertoli cells are testicular cells that are thought to play a major role in sperm production. Sertoli cells secrete a number of proteins including transport proteins, hormones and growth factors, enzymes which regulate germinal cell development and other biological processes related to reproduction (Griswold, Int. Rev. Cytol., 133-156, 1988). They secrete the peptide hormone inhibin B, an important negative feedback signal to the anterior pituitary. They assist in spermiation (the final detachment of the mature spermatozoa from the Sertoli cell into the lumen) by releasing plasminogen activator which produces proteolytic enzymes. The data show that T-type calcium channels are expressed on immature rat Sertoli cells according to Lalevee et al., 1997. The intimate juxtaposition of the developing germ cells with the Sertoli cells suggests that the Sertoli cells may indeed pay a role in supporting and nurturing the gametes. While the role of T-type calcium channels is not well documented, it is believed that they may be important in the release of nutrients, inhibin B, and/or plasminogen activator and thus may impact sperm production. According to researchers, the inhibition of T-type calcium channels in sperm during gamete interaction inhibits zona pellucida-dependent Ca²⁺ elevations and inhibits acrosome reactions, thus directly linking sperm T-type calcium channels to fertilization. See Arnoult et al., 1996.

Likewise, tremor can be controlled through the basal ganglia and the thalamus, regions in which T type calcium channels are strongly expressed (Talley et al., supra). T-type calcium channels have been implicated in the pathophysiology of tremor since the anti-epileptic drug ethosuximide is used for treating tremor, in particular, tremor associated with Parkinson's disease, essential tremor, or cerebellar disease (U.S. Pat. No. 4,981,867; D. A. Prince).

T-type calcium channels also facilitate insulin secretion by enhancing the general excitability of these cells. Therefore, T-type calcium channels may be therapeutic targets in hypo- and hyperinsulinemia (A. Bhattacharjee et al., Endocrinology, vol. 138(9), pp. 3735-40, 1997). A direct link between T-type calcium channel activity and steroidogenesis has been suggested (M. F. Rossier et al., 1996).

Cellular calcium homeostasis plays an essential part in the physiology of nerve cells. The intracellular calcium concentration is about 0.1 uM compared with 1 mM outside the nerve cell. This steep concentration gradient (×10,000) is regulated primarily by voltage-gated calcium channels. Several pathologies of the central nervous system involve damage to or inappropriate function of voltage-gated calcium channels. In cerebral ischaemia (stroke) the channels of neurons are kept in the open state by prolonged membrane depolarization, producing a massive influx of calcium ions. This, in turn activates various calcium/calmodulin dependent cellular enzyme systems, e.g. kinases, proteases and phospholipases. Such prolonged activation leads to irreversible damage to nerve cells.

Certain diseases, such as Lambert-Eaton Syndrome, involve autoimmune interactions with calcium channels. The availability of the calcium channel subunits makes possible immunoassays for the diagnosis of such diseases. An understanding of them at the molecular level will lead to effective methods of treatment.

As well, there is a need for a better understanding of the structure and function of calcium channels, which, in turn would permit identification of substances that, in some manner, modulate the activity of calcium channels and that have potential for use in treating such disorders. That mutations of several channel proteins have been shown to be a causative factor in neurological disorders, is well known, thereby making the calcium channel subunits target for therapeutic interventions. See, e.g., Marais, supra and Burgess and Noebels, (1999) Epilepsy Res., 36:111-122.

An understanding of the pharmacology of compounds that interact with calcium channels in other organ systems, such as the central nervous system (“CNS”), will greatly aid in the rational design of compounds that specifically interact with the specific subtypes of human calcium channels to have desired therapeutic effects, such as in the treatment of neurodegenerative and cardiovascular disorders. Such an understanding together with the ability to rationally design therapeutically effective compounds have been hampered by an inability to independently determine the types of human calcium channels and the molecular nature of individual subtypes, particularly in the CNS, and by the unavailability of pure preparations of specific channel subtypes to use for evaluation of the specificity of calcium channel-effecting compounds. Thus, the identification of nucleic acid molecules encoding human calcium channel subunits coupled with the use of such molecules for expression of the encoded calcium channel subunits subsequent use in of the functional calcium channels would aid in screening and design of therapeutically effective compounds.

A number of compounds useful in treating various diseases in animals, including humans, are thought to exert their beneficial effects by modulating functions of voltage-gated calcium channels. Many of these compounds bind to calcium channels and block, or reduce the rate of influx of calcium into cells in response to depolarization of the inside and outside of the cells. An understanding of the pharmacology of compounds that interact with calcium channels, and the ability to rationally design compounds that will interact with calcium channels to have desired therapeutic effects, depends upon the understanding of the structure of calcium subunits and the genes that encode them. The identification and study of tissue specific subunits allows for the development of therapeutic compounds specific for pathologies of those tissues.

However, there is a paucity of understanding of the pharmacology of compounds which interact with calcium channels. This paucity of understanding, together with the limited knowledge in the art of the human calcium channel types, the molecular nature of the human calcium channel subtypes, and the limited availability of pure preparations of specific calcium channel subtypes to use for evaluating the efficacy of calcium channel-modulating compounds has hampered the rational testing and screening of compounds that interact with the specific subtypes of human calcium channels to have desired therapeutic effects.

While a number of pharmacological blockers have differential effects on T type calcium currents expressed in different cell types as noted supra, there are no known specific blockers of the T-type class of calcium channel. It is believed that the differential sensitivity of T-type currents to antagonists may be due to different subunit structure (Perez-Reyes, 1998) as well as cellular environments. T-type calcium channel alpha subunit genes, like the genes for HVA channels, reveal alternative splicing (Lee et al., 1999 Biophys J 76:A408). Extracellular and intracellular loops of individual T-type calcium channel clones also show marked diversity amongst themselves and even less homology to HVA channels.

Examples of conventional putative calcium channel blockers include dihydropyridines such as nifedipine, nitrendipine, nicardipine, nimodipine, niludipine, riodipine (ryosidine) felodipine, darodipine, isradipine, (+)Bay K 8644, (−)202-791, (+)H 160/S1, PN 200-110 and nisoldipine. Other examples of the calcium channel blocker include Kurtoxin, benzothiazepine, such as diltiazem (dilzem) and TA 3090 and phenylalkylamine, such as verapamil (isoptin), desmethoxyverapamil, methoxy verapamil (D-600, gallopamil or (−)D-888), prenylamine, fendiline, terodiline, caroverine, perhexiline.

In view of the above, pharmacological modulation of T-type calcium channels' function is very important and therapeutic moieties capable of modulating T-type currents will find tremendous use in the practice of medicine, i.e., calcium channel blockers for the treatment of epilepsy, hypertension, and angina pectoris etc. Unfortunately, as noted above, conventional medicine and its use of conventional calcium channels blockers for the treatment of a wide variety of calcium channels mediated diseases is not very effective. Importantly, such intervention is not yet available for calcium channels in electrically non-excitable cells. This deficiency likely reflects the fact that the mechanism by which calcium entry occurs has not been clearly identified.

Recent studies that demonstrated the association of mutations in calcium channel genes (α₁ and β genes) with inherited and acquired diseases further underlined the importance of calcium channels and have created a new field of research aimed at understanding and controlling these “channelopathies” (Miller, supra).

Various efforts have been made to obtain sequences of calcium channel subunit genes, such as the human (α₂)-subunit gene (Ellis et al., Science 241(4873):1661-[1988]; Williams et al., Neuron, 8(1):71-84 [1992]; Ellis et al. U.S. Pat. No. 5,686,241; and Harpold et al., U.S. Pat. No. 5,792,846), and its murine (GenBank Accession ##U73483-U73487), rat (GenBank Accession #M86621), porcine (GenBank Accession #M21948), and rabbit orthologs (GenBank Accession #AF077665).

Significantly, the development of new therapeutic strategies against, and the creation of new analytical tools for a better understanding of diseases characterized by aberrant voltage regulated calcium influx are greatly desired.

Because T-type channels appear to be associated with a variety of key functions, cells that express T-channels and assays using such cells will have utility in the identification of compounds effective in modulating a T-type channel, and thus will find use in the treatment of a variety of disorders, disease and conditions effecting both humans and animals. Compounds identified thereby will be candidates for use in the treatment of disorders and conditions associated with T-channel activity in humans and animals. Such activities include, but are not limited to, those involving a role in muscle excitability, secretion and pacemaker activity, Ca²⁺ dependent burst firing, neuronal oscillations, and potentiation of synaptic signals, for improving arterial compliance in systolic hypertension, or improving vascular tone, such as by decreasing vascular welling, in peripheral circulatory disease, and others. Other disorders include, but are not limited to hypertension, cardiovascular disorders, including but not limited to: myocardial infarct, cardiac arrhythmia, heart failure and angina pectoris; neurological disorders, such as schizophrenia, epilepsy and depression, peripheral muscle disorders, respiratory disorders and endocrine disorders.

Consequently, the discovery of the herein disclosed sequences of murine α_{1H} subunits will allow for the development of therapeutic compounds specific for the pathologies noted above thereby satisfying a long-sought need for such therapies and tools.

### SUMMARY OF THE INVENTION

The present invention is based on the discovery of a novel low-voltage calcium channel α_{1H} subunit (Caᵥ 3.2) from three strains of rats—Sprague-Dawley (S-D), Spontaneous Hypertensive (SHR) and Wystar-Kyoto (WKY). Importantly, the amino acid sequence encoded by each of the nucleic acid sequences derived from SHR and WKY are identical whereas the amino acid sequence encoded by the nucleic acid sequence derived from the S-D differs from that of the SHR and WKY at position 2188. These calcium channel subunits of the invention are the major pathway for regulating influx of Ca²⁺ into cells and play critical roles in diverse cellular processes such as electrical excitability and contraction, hormone secretion, enzyme activity, and gene expression.

The invention and its use is based, in part, on the fact that the murine calcium channel α_{1H} subunit (Caᵥ 3.2) is closely related to a mammalian calcium channel α_{1H} subunit (Caᵥ 3.2). It is also based on the tissue distribution of the exact matches, related sequences or variants of SEQ ID NOS:1-6 which may be found in heart, kidney, liver, brain and endocrine tissues.

The use of the herein disclosed calcium channel α_{1H} subunit, and of the nucleic acid sequences which encode it, is also based on the amino acid and structural homologies between the herein disclosed α_{1H} subunit and the other known T-type calcium channel subunits as well as on the known associations and functions of T-type calcium channels in general. The timing of and amount of expression of any one or more of the polypeptides of the invention, calcium channel α_{1H} subunit of SEQ ID NOS:2, 4 and 6 is implicated in various diseases characterized by a dysfunctional or aberrant expression/activity of a T-type calcium channel, in particular, an α_{1H} subunit. Given the tissue distribution, the novel T-type calcium channel α_{1H} subunit(s) in this application are likely involved in signal transduction pathways related to cardiac, renal, endocrine and neuronal cell activity.

An illustrative nucleic acid molecule containing a sequence that encodes the α_{1H} polypeptide has the nucleotide sequence of SEQ ID NO:1 of 7426 nucleotides, of which the coding sequence encompasses nucleotides 50 to 7129. This sequence is designated herein as α_{1H}—SHR. The coding sequence contained within SEQ ID NO:1 is 7080 nucleotides (nts). The encoded polypeptide has the amino acid sequence as set forth in SEQ ID NO:2.

Another illustrative nucleic acid molecule containing a sequence that encodes the α_{1H} polypeptide has the nucleotide sequence of SEQ ID NO:3 of which the coding sequence encompasses nucleotides 56 to 7135. This sequence is designated herein as α_{1H}—WKY. The coding sequence contained within SEQ ID NO:3 is 7080 nts. The encoded polypeptide has the amino acid sequence as set forth in SEQ ID NO:4. Thus, the α_{1H}—WKY nucleotide sequence described herein encodes a polypeptide that is 2359 amino acids.

Yet another illustrative nucleic acid molecule containing a sequence that encodes the α_{1H} polypeptide has the nucleotide sequence of SEQ ID NO:5 of 7277 nucleotides, of which the coding sequence encompasses nucleotides 50 to 7129. This sequence is designated herein as α_{1H}—S-D. The coding sequence contained within SEQ ID NO:5 is 7080 nts. The encoded polypeptide has the amino acid sequence as set forth in SEQ ID NO:6.

In another aspect, the invention provides nucleic acid molecule(s) comprising a nucleotide sequence which is complementary to that of SEQ ID NOS:1, 3, or 5 or complementary to a sequence having at least 90% identity to said sequence or a fragment of said sequence. The complementary sequence may be a DNA sequence which hybridizes with, for example, SEQ ID NO:1 or hybridizes to a portion of that sequence having a length sufficient to inhibit the transcription of the complementary sequence. The complementary sequence may be a DNA sequence which can be transcribed into an mRNA being an antisense to the mRNA transcribed from SEQ ID NO:1 or into an mRNA which is an antisense to a fragment of the mRNA transcribed from SEQ ID NO:1 which has a length sufficient to hybridize with the mRNA transcribed from SEQ ID NO:1, so as to inhibit its translation. The complementary sequence may also be the mRNA or the fragment of the mRNA itself.

Considering the high degree (>90%) of sequence homology in the primary sequence between the reference α_{1H} sequence GenBank accession #AF211189 and the corresponding human α_{1H} subunit (AF073931) and the novel sequences disclosed herein, it is believed that compositions comprising the novel sequences or biologically active fragments or derivatives thereof may be administered to a subject to treat or prevent a pathological disorder characterized by a dysfunctional T-type calcium channel subunit. As such, the novel proteins of the invention may find use, inter alia, in treating a number of (x1H subunit mediated pathologies including epilepsy, colorectal cancers, gastric cancers, acute myelogenous leukemias as well as lung and breast cancers. See, for example, McRory, et al., J. Biol. Chem., 276 (6), 3999-4011 (2001).

The present invention further provides nucleic acid molecule comprising a nucleotide sequence which encode the amino acid sequences of SEQ ID NOS:2, including fragments and homologues of the amino acid sequences. Due to the degenerative nature of the genetic code, a plurality of alternative nucleic acid sequences beyond those depicted in SEQ ID NO:1, can code for the amino acid sequences of the invention. Consequently, those alternative nucleic acid sequences which code for the same amino acid sequences coded by the sequence of SEQ ID NO:1 are also included in the scope of the present invention.

The present invention also relates, in part, to an expression vector and host cells comprising nucleic acids encoding an α_{1H} subunit of the invention. Such transfected host cells are useful for the production and recovery of α_{1H}. The present invention also encompasses purified α_{1H}. The present invention still further provides pharmaceutical compositions comprising, as an active ingredient, nucleic acid molecules encoding a functional α_{1H} protein/polypeptide or antibodies specific thereto, fragments or variants thereof or a therapeutic composition identified via use of the herein disclosed nucleic acid molecules e.g., inhibitors of a T-type calcium channel α_{1H} subunit which can be used in the prevention or treatment of conditions or diseases noted below.

In another aspect, the invention provides a protein or polypeptide comprising an amino acid sequence encoded by any of the above nucleic acid sequences. In one embodiment, the polypeptide corresponding to α_{1H} comprises the amino acid sequence of SEQ ID NO:2 ((SHR). In another embodiment the polypeptide corresponds to α_{1H} (WKY) and comprises the amino acid sequence of SEQ ID NO:4. Yet another polypeptide corresponds to α_{1H} (S-D) and comprises the amino acid sequence of SEQ ID NO:6. Fragments of the above amino acid sequences of sufficient length coded by the above fragments of the nucleic acid sequences, as well as homologues of the above amino acid sequences in which one or more of the amino acid residues has been substituted by conservative or non-conservative substitution) added, deleted, or chemically modified are also within the scope of the invention.

The deletions, insertions and modifications should be in regions, or adjacent to regions, wherein the novel isoforms differs from the reference sequence, but maintains its ability to regulate voltage gated calcium influx. Applicants appreciate that a skilled artisan will be able to modify the novel isoforms or fragments thereof by addition, deletions or substitutions of amino acids (derivative product/polypeptide). Consequently, homologues of the α_{1H} variants which are derivated from the reference α_{1H} sequence e.g., α_{1H} (SEQ ID NO:1, 3 or 5) by changes (deletion, addition, substitution) are also a part of the present invention, wherein said derivatized sequence is functionally equivalent to the novel sequences detailed herein, i.e., ability to modulate voltage-gated calcium influx etc.

Medicaments for treating α_{1H} subunit mediated disorders in human or animals identified via the use of the herein disclosed sequences, are also a part of the invention. Such medicaments will find use in the treatment of diseases and pathological conditions where a therapeutically beneficial effect may be achieved by correcting abnormal calcium influx. Typically, these are diseases wherein α_{1H} or other auxiliary subunit proteins of the calcium channel plays a role in the etiology of the disease, i.e. aberrant (excessive or insufficient voltage regulated calcium influx) cause or are a result of the disease.

The invention further features a method for identifying a candidate pharmacological agent useful in the treatment of diseases associated with increased or decreased voltage regulated calcium influx mediated by a human T-type calcium channel α_{1I} subunit isoform of the invention.

Compounds identified by any of the herein disclosed methods are also within the scope of the invention.

Thus, in accordance with an aspect of the invention, suitable host cells expressing functional LVA channels, such as an α_{1H} subunit of the invention, preferably those encoding SEQ ID NOS:2, 4 or 6, will find use in identifying compounds that are candidates for treatment of disorders associated with a dysfunctional T-type calcium channel or normal functioning T-type channels impacting a disease state. Representative disorders amenable to treatment by compounds identified via use of the herein disclosed sequences include treatment of cardiovascular, such as angina, vascular, such as hypertension, and urologic, hepatic, reproductive, adjunctive therapies for reestablishing normal heart rate and cardiac output following traumatic injury, heart attack and other cardiac injuries; treatments of myocardial infarct (MI), post-MI and in an acute setting. Endocrionology diseases especially hyper aldosteronism and diseases of the central nervous system are also amenable to treatment by compounds identified using any one or more of the ovel sequences disclosed herein.

Other compounds that interact with LVA, particularly T-type, calcium channels, may be effective for increasing cardiac contractile force, such as measured by left ventricular end diastolic pressure, and without changing blood pressure or heart rate. Alternatively, some compounds may be effective to decrease formation of scar tissue, such as that measured by collagen deposition or septal thickness, and without cardiodepressant effects.

The herein disclosed assays may also be used to
(a) identify compounds useful in regulating vascular smooth muscle tone, either vasodilating or vasoconstricting in:
   (i) treatments for reestablishing blood pressure control, e.g., following traumatic injury, surgery or cardiopulmonary bypass, and in prophylactic treatments designed to minimize cardiovascular effects of anesthetic drugs;
   (ii) treatments for improving vascular reflexes and blood pressure control by the autonomic nervous system;
(b) identify compounds useful in treating urological disorders, e.g., treating and restoring renal function following surgery, traumatic injury, uremia and adverse drug reactions; treating bladder dysfunctions; and uremic neuronal toxicity and hypotension in patients on hemodialysis; reproductive disorders,
(c) identify compounds useful in treating:
   (i) disorders of sexual function including impotence;
   (ii) alcoholic impotence (under autonomic control that may be subject to T-channel controls);
   (iii) hepatic disorders for identifying compounds useful in treating and reducing neuronal toxicity and autonomic nervous system damage resulting from acute over-consumption of alcohol; neurologic disorders for identifying compounds useful in treating:
      (a) epilepsy and diencephalic epilepsy;
      (b) Parkinson's disease;
      (c) aberrant temperature control, such as, abnormalities of shivering and sweat gland secretion and peripheral vascular blood supply;
      (d) aberrant pituitary and hypothalamic functions including abnormal secretion of noradrenalin, dopamine and other hormones; for respiratory such as in treating abnormal respiration, e.g., post-surgical complications of anesthetics; and endocrine disorders, for identifying compounds useful in treating aberrant secretion of hormones including e.g., possible treatments for overproduction of insulin, thyroxin, adrenalin, and other hormonal imbalances.

In a broad aspect, the invention provides a method for screening for compounds which modulate the activity of T-type voltage-gated calcium channels. The method involves providing a cell transformed with a DNA expression vector comprising a cDNA sequence encoding a T-type α_{1H} subunit of a voltage-gated calcium channel, e.g., a murine α_{1H} subunit of a voltage-gated calcium channel, the cell comprising additional calcium channel subunits necessary and sufficient for assembly of a functional low-voltage-gated calcium channel. The cell is contacted with a test compound and agonistic or antagonistic action of the test compound on the reconstituted calcium channels is determined.

Without intending to limit the type or source of host cell, in yet another preferred embodiment, the host cell is eukaryotic.

In another aspect, a method of the invention proposes that the eukaryotic cell that expresses a heterologous calcium channel is in a solution containing a test compound and a calcium channel selective ion, the cell membrane is depolarized, and current flowing into the cell is detected. If the test compound is one that modulates calcium channel activity, the current that is detected is different from that produced by depolarizing the same or a substantially identical cell in the presence of the same calcium channel-selective ion but in the absence of the compound (control cell). Preferably, prior to the depolarization step, the cell is maintained at a holding potential which substantially inactivates calcium channels which are endogenous to the cell. As well, in certain preferred embodiments, the cells are mammalian cells, most preferably HEK cells, or amphibian oocytes.

Thus, in accordance with the above, there is provided a method for screening test compounds for modulating calcium channel activity, comprising:
a) providing:
   i) the test compound;
   ii) a calcium channel selective ion;
   iii) a control cell; and
   iv) a host cell expressing heterologous nucleic acid sequences encoding: a functional calcium channel α_{1H} subunit; preferably one having the amino acid sequence as set forth in one of SEQ ID NOS: 2, 4 or 6 or a biologically equivalent/active fragment thereof;
b) contacting the host cell with the test compound and with the molecule to produce a treated host cell;
c) depolarizing the cell membrane of the treated host cell under conditions such that the molecule enters the cell through a functional calcium channel; and
d) detecting a difference between current flowing into the treated host cell and current flowing into a control cell, thereby identifying the test compound as a compound capable of modulating calcium channel activity.

The method further comprises, prior to the depolarizing, maintaining the treated host cell at a holding potential that substantially inactivates endogenous calcium channels. In another preferred embodiment, the method further comprises, prior to or simultaneously with the step of contacting the host cell with the test compound, contacting the host cell with a calcium channel agonist, wherein the test compound is tested for activity as an antagonist.

Alternative embodiments propose a transcription based assays for identifying compounds that modulate the activity of calcium channels (see, U.S. Pat. Nos. 5,436,128 and 5,401,629), in particular calcium channels that contain an α_{1H} subunit.

Other reporter based assays may include the use of a dye which coordinate Ca²⁺. The method provides (i) incubating recombinant cells of the invention (those expressing a function calcium channel α_{1H} subunit) with (1) a dye which has acid groups which can coordinate Ca²⁺ and which undergoes a spectral shift when coordinated to Ca²⁺ and (2) a compound with unknown effect; (ii) stimulating Ca²⁺ influx into the cell; and (iii) monitoring the spectral characteristics of the dye in the recombinant cells. These spectral characteristics will change as calcium is bound to the dye. Because calcium will bind to (be coordinated by) the dye in proportion to the concentration of calcium in the activated cell, the change in spectral characteristics of the dye will be a measure of the calcium concentration within the cell. If the compound is a T-type channel selective inhibitor then the absorbance or fluorescent emission of the uncoordinated dye (A) will be different than the absorbance or fluorescent emission of the Ca²⁺-coordinated dye (A2) because the inhibitor will have suppressed calcium entry into the cell. In preferred embodiments, the DNA is one of SEQ ID NOS:1, 3 or 5.

Other assays formats, well known to one skilled in the art, for identifying calcium channel modulators, in particular T-type calcium channels may also be used.

The invention further provides diagnostic kits for the detection of naturally occurring α_{1H} sequences and provides for the use of purified α_{1H} as a positive control and to produce anti-α_{1H} antibodies. These antibodies may be used to monitor α_{1H} expression conditions or diseases associated with aberrant expression or mutated α_{1H}. Alternatively, the sequences of the invention may be used to detect mutations within a gene encoding a T-type α_{1H} subunit.

Thus, an aspect of the invention provides antibodies specific for one or more of the novel proteins of the invention, which may be used in identifying corresponding genes in humans having a sequence of amino acids substantially similar to that one the sequence which was used to generate said antibody. Consequently, antibodies specific for a protein of the invention will find use for identifying corresponding proteins in humans, e.g. western blot etc. Thus, such antibodies may be useful for diagnostic purposes in humans. Methods for generating antibodies are well known.

The immunoglobulins that are produced using the calcium channel subunits or purified calcium channels as immunogens have, among other properties, the ability to specifically and preferentially bind to and/or cause the immunoprecipitation of a human calcium channel or a subunit thereof which may be present in a biological sample or a solution derived from such a biological sample. Such antibodies may also be used to selectively isolate cells that express calcium channels that contain the subunit for which the antibodies are specific.

The α_{1H} polynucleotide sequence, oligonucleotides, fragments, portions or antisense molecules thereof, may be used in diagnostic assays to detect and quantify levels of α_{1H} mRNA in cells and tissues. For example, α_{1H} polynucleotides, or fragments thereof, may be used in hybridization assays of body fluids or biopsied tissues to detect the level of α_{1H} expression.

Thus, an aspect of the invention features methods for (i) detecting the level of the transcript (mRNA) of said α_{1H} subunit or a variant product (SEQ ID NO:1, 3 or 5, or fragments thereof) in a body fluid sample, or in a specific tissue sample, for example by use of probes comprising all or parts of the nucleotide sequences disclosed herein; (ii) detecting levels of expression of said subunit in tissue, e.g. by the use of antibodies capable of specifically reacting with the gene products of the nucleotide sequences of the invention or biologically equivalent fragments thereof. Detection of the level of the expression of a variant product(s) of the invention in particular as compared to that of the reference sequence from which it was varied or compared to other variant sequences all varied from the same reference sequence may be indicative of a plurality of physiological or pathological conditions. Quantifying normal levels of the target gene or its encoded gene product are well known to a skilled artisan.

The probes o f the invention, in turn, may be used to detect and quantify the level of transcription of a corresponding human α_{1H} channel subunit in a human for diagnostic and therapeutic purposes. The method, according to this latter aspect, for detecting a nucleic acid sequence which encodes a human T-type calcium channel α_{1H} subunit isoforms in a biological sample, comprises the steps of:
(a) providing a probe comprising at least one of the nucleic acid sequences disclosed herein;
(b) contacting the biological sample with said probe under conditions allowing hybridization of nucleic acid sequences thereby enabling formation of hybridization complexes;
(c) detecting hybridization complexes, wherein the presence of the complex indicates the presence of nucleic acid sequence encoding the α_{1H} subunit or an isoform thereof in the biological sample.

The methods as described above are qualitative, i.e. indicate whether the transcript or gene product is present in or absent from the sample. The method can also be quantitative, by determining the level of hybridization complexes and/or protein/antibody complex and then calibrating said levels to determining levels of transcripts or antibody complexes of the desired variant in the sample. Both qualitative and quantitative determination methods can be used for diagnostic, prognostic and therapy planning purposes.

The nucleic acid sequence used in the above method may be a DNA sequence, an RNA sequence, etc; it may be a coding or a sequence or a sequence complementary thereto (for respective detection of RNA transcripts or coding-DNA sequences). By quantization of the level of hybridization complexes and calibrating the quantified results it is possible also to detect the level of the transcript in the sample.

Methods for modulating the activity of ion channels by contacting the calcium channels with an effective amount of the above-described antibodies are also provided.

Methods for treating subjects suffering from or at risk of being afflicted with a pathology/disease characterized by aberrant voltage regulated calcium influx using compounds identified by the methods of the present invention are also embraced by the invention. The disease status can be characterized as aberrant—excessive or insufficient voltage regulated calcium influx relative to normal.

Also included are methods for diagnosing LVA calcium channel-mediated, particularly T-type channel-mediated, disorders. Methods of diagnosis will involve detection of aberrant channel expression or function, such altered amino acid sequences, altered pharmacological profiles and altered electrophysiological profiles compared to normal or wild-type channels. Such methods typically can employ antibodies specific for the altered channel or nucleic acid probes to detect altered genes or transcripts.

In another aspect, the present invention relates to diagnostic screening techniques useful for the identification of mutations within the α_{1I} encoding (Caᵥ3.3) gene that is involved in neuronal disorders. The proposed method will involve detection of a species of α_{1H} sequence via a Northern. Southern or western blot using any one or more sequences of the invention.

Thus, initial identification of mutations responsible for such conditions can be made, for example, by producing cDNA from the mRNA of an individual suffering from a neuronal disorder (e.g., epilepsy). The sequence of nucleotides in the cDNA is then determined by conventional techniques. This determined sequence is then compared to the wild-type sequence available in the public database. Differences between the determined cDNA sequence, and that disclosed in the public database, GeneBank Accession #AF290213, are candidate deleterious mutations. Following identification and characterization, oligonucleotides can be designed for the detection of specific mutants. Alternatively, a α_{1H} gene can be isolated from the genome of a patient and directly examined for mutations by such techniques as restriction mapping or sequencing.

To determine whether such mutations are responsible for the diseased phenotype, experiments can be designed in which the defective gene carrying the identified mutation is introduced into a cell system expressing a complement of components sufficient for the production of functional neuronal low-voltage-gated calcium channels. The ability of the mutant α_{1H} sequence to function as a calcium channel can be assessed using conventional techniques, such as the ones described above.

Other aspects of the present invention are presented in the accompanying claims and in the following description and drawings. These aspects are presented under separate section headings. However, it is to be understood that the teachings under each section are not necessarily limited to that particular section heading.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 details the intracellular recording or patch-clamp recording used to quantitate changes in electrophysiology of cells for the SHR channels.

### DETAILED DESCRIPTION OF THE INVENTION

Before the present proteins, nucleotide sequences, and methods are described, it is to be understood that the present invention is not limited to the particular methodologies, protocols, cell lines, vectors, and reagents described, as these may vary. It is also understood that the terminology used herein is for the purpose of describing particular embodiments only, and is not to limit the scope of the present invention.

The singular forms “a,” “an,” and “the” include plural reference unless the context clearly dictates otherwise.

All technical and scientific terms used herein have the same meanings as commonly understood by one of ordinary skill in the art to which this invention pertains. The practice of the present invention will employ, unless otherwise indicated, conventional techniques of protein chemistry and biochemistry, molecular biology, microbiology and recombinant DNA technology, which are within the skill of the art. Such techniques are explained fully in the literature.

Although any machines, materials, and methods similar or equivalent to those described herein can be used to practice or test the present invention, the preferred machines, materials, and methods are now described. All patents, patent applications, and publications mentioned herein, whether supra or infra, are each incorporated by reference in its entirety.

While the description details various embodiments encompassing the nucleic acid molecule of SEQ ID NO:1 and the encoded protein of SEQ ID NO:2, including variants and fragments thereof, the same description applies equally to the nucleic acid molecules of SEQ ID NOS:3 and 5, and the encoded proteins of SEQ ID NOS:4 and 6, including various fragments, and variants thereof. For example, just as the rat T-type calcium channel α_{1H} subunit encoding nucleic acid of SEQ ID NO:1 is “isolated”, so is the rat T-type calcium channel α_{1H} subunit encoding nucleic acid molecule of SEQ ID NOS:3 and 5 etc.

Glossary

In the following description and claims use will be made, at times, with a variety of terms, and the meaning of such terms as they should be construed in accordance with the invention is as follows:

In the following commentary, a “gene” refers to a nucleic acid molecule whose nucleotide sequence codes for a polypeptide molecule. Genes may be uninterrupted sequences of nucleotides or they may include such intervening segments as introns, promoter regions, splicing sites and repetitive sequences. A gene can be either RNA or DNA. A preferred gene is one that encodes the invention protein.

The present invention relates to various novel murine T-type calcium channel subunits, and to the use of the nucleic acid and amino acid sequences in the study, diagnosis, prevention and treatment of diseases mediated by a dysfunctional calcium channel α_{1H} subunit.

The polynucleotide sequence encoding one or more of the herein disclosed α_{1H} subunit were identified as outlined in the Examples infra.

The present invention and the use of the α_{1H} subunit sequences identified herein, and of the nucleic acid sequences which encode it, is based, in part, on the amino acid homology between the murine α_{1H} subunit and the corresponding human protein. It is also based on the tissue distribution of variants, closely related or exact cDNA sequences in (describe tissue distribution, if known).

The murine α_{1H} SHR subunit polynucleotide sequence, oligonucleotides, fragments, portions or antisense thereof, may be used in diagnostic assays to detect and quantify levels of α_{1H} SHR subunit mRNA in cells and tissues, genomic as well as mutated sequences. For example, α_{1H} SHR subunit polynucleotides, or fragments thereof, may be used in hybridization assays of body fluids or biopsied tissues to detect the level of α_{1H} SHR subunit expression. The invention further provides for the use of purified α_{1H} SHR subunit as a positive control and to produce anti-α_{1H} SHR subunit antibodies. These antibodies may be used to monitor α_{1H} SHR subunit expression in conditions or diseases associated with dysfunctional or aberrant levels of calcium ions.

The present invention also relates, in part, to an expression vector and host cells comprising nucleic acids encoding α_{1H} SHR subunit. Such transfected host cells are useful for the production and recovery of α_{1H} SHR subunit. The present invention also encompasses purified α_{1H} SHR subunit.

The invention further provides for methods for treatment of conditions or diseases associated with over-expression of α_{1H} subunit by the delivery of effective amounts of antisense molecules, including peptide nucleic acids, or inhibitors of α_{1H} subunit for the purpose of diminishing or correcting aberrant calcium channel activity.

The invention also provides pharmaceutical compositions comprising vectors containing antisense molecules or inhibitors of α_{1H} SHR which can be used in the prevention or treatment of conditions or diseases including, but not limited to, epilepsy, pain, cardiac arrhythmia, sleep disorders etc that are mediated by a deficient or dysfunctional T-type calcium channel subunit. Thus, for example, specific α_{1H} SHR inhibitors can be used to prevent aberrant calcium currents.

“Nucleic acid sequence” as used herein refers to an oligonucleotide, nucleotide or polynucleotide sequence, and fragments or biologically equivalent portions thereof, and to DNA or RNA of genomic or synthetic origin which may be single- or double-stranded, and represent the sense or antisense strand. Similarly, amino acid sequence as used herein refers to an oligopeptide, peptide, polypeptide or protein sequence. “Peptide nucleic acid” as used herein refers to a molecule which comprises an antisense oligomer to which an amino acid residue, such as lysine, and an amino group have been added. These small molecules, also designated anti-gene agents, stop transcript elongation by binding to their complementary (template) strand of DNA (Nielsen P. E. et al (1993) Anticancer Drug Des 8:53-63). Thus, “nucleotide sequence of the present invention” and “amino acid sequence of the present invention” and grammatical equivalents thereof refer respectively to any one or more nucleotide sequences presented or discussed herein and to any one or more of the amino acid sequences presented or discussed herein. Also, and as used herein, “amino acid” refers to peptide or protein sequence and may refer to portions thereof. In addition, the term “amino acid sequence of the present invention” is synonymous with the phrase “polypeptide of the present invention”. Also the term “nucleotide sequence of the present invention” is synonymous with the phrase “poly-nucleotide sequence of the present invention”.

As used herein, α_{1H} refers to the amino acid sequence of α_{1H} from a rat, in a naturally occurring form or from any source, whether natural, synthetic, semi-synthetic or recombinant. As used herein, “naturally occurring” refers to a molecule, nucleic acid or amino acid sequence, found in nature.

The present invention also encompasses α_{1H} variants. A preferred α_{1H} variant is one having at least 80% amino acid sequence similarity, a more preferred α_{1H} variant is one having at least 90% amino acid sequence similarity and a most preferred α_{1H} variant is one having at least 95% amino acid sequence similarity to the α_{1H} amino acid sequence (SEQ ID NO:2). A “variant” of α_{1H} SHR may have an amino acid sequence that is different by one or more amino acid “substitutions”. The variant may have “conservative” changes, wherein a substituted amino acid has similar structural or chemical properties, eg, replacement of leucine with isoleucine. More rarely, a variant may have “nonconservative” changes, eg, replacement of a glycine with a tryptophan. Similar minor variations may also include amino acid deletions or insertions, or both. Guidance in determining which and how many amino acid residues may be substituted, inserted or deleted without abolishing biological or immunological activity may be found using computer programs well known in the art, for example, DNASTAR software.

The term “biologically active” refers to a α_{1H} sequence having structural, regulatory or biochemical functions of the naturally occurring α_{1H}. Likewise, “immunologically active” defines the capability of the natural, recombinant or synthetic α_{1H} subunit, or any oligopeptide thereof, to induce a specific immune response in appropriate animals or cells and to bind with specific antibodies. The term “derivative” as used herein refers to the chemical modification of a α_{1H} encoding sequence or the encoded α_{1H} subunit. Illustrative of such modifications would be replacement of hydrogen by an alkyl, acyl, or amino group. An α_{1H} encoding nucleotide sequence derivative would encode a polypeptide which retains essential biological characteristics of a T-type calcium channel α_{1H} protein g subunit such as, for example, to for, a functional calcium channel.

As used herein, the term “purified” refers to molecules, either nucleic or amino acid sequences, that are removed from their natural environment and isolated or separated from at least one other component with which they are naturally associated.

The α_{1H} SHR Coding Sequences

The nucleic and deduced amino acid sequences of α_{1H} subunit, e.g., α_{1H} SHR are shown in SEQ ID NOS:1 and 2 respectively. In accordance with the invention, any nucleotide sequence which encodes the amino acid sequence of α_{1H} SHR can be used to generate recombinant molecules which express α_{1H} SHR.

Methods for DNA sequencing are well known to a skilled artisan and may employ such enzymes as the Klenow fragment of DNA polymerase I Sequenase.RTM. (US Biochemical Corp, Cleveland Ohio)), Taq polymerase (Perkin Elmer, Norwalk Conn.), thermostable T7 polymerase (Amersham, Chicago Ill.), or combinations of recombinant polymerases and proofreading exonucleases such as the ELONGASE Amplification System marketed by Gibco BRL (Gaithersburg Md.). As well, methods to extend the DNA from an oligonucleotide primer annealed to the DNA template of interest have been developed for both single-stranded and double-stranded templates. Chain termination reaction products were separated using electrophoresis and detected via their incorporated, labelled precursors. Recent improvements in mechanized reaction preparation, sequencing and analysis have permitted expansion in the number of sequences that can be determined per day. Preferably, the process is automated with machines such as the Hamilton Micro Lab 2200 (Hamilton, Reno Nev.), Peltier Thermal Cycler (PTC200; MJ Research, Watertown Mass.) and the ABI Catalyst 800 and 377 and 373 DNA sequencers (Perkin Elmer).

The quality of any particular cDNA library may be determined by performing a pilot scale analysis of the cDNAs and checking for percentages of clones containing vector, lambda or E. coli DNA, mitochondrial or repetitive DNA, and clones with exact or homologous matches to public databases.

Extending the Polynucleotide Sequence:

The polynucleotide sequence of α_{1H} SHR (SEQ ID NO:1) may be extended utilizing partial nucleotide sequence and various methods known in the art to detect upstream sequences such as promoters and regulatory elements. Gobinda et al (1993; PCR Methods Applic 2:318-22) disclose “restriction-site polymerase chain reaction (PCR)” as a direct method which uses universal primers to retrieve unknown sequence adjacent to a known locus. According to the process, initially, a genomic DNA is amplified in the presence of primer to a linker sequence and a primer specific to the known region. Thereafter, the amplified sequences are subjected to a second round of PCR with the same linker primer and another specific primer internal to the first one. Products of each round of PCR are transcribed with an appropriate RNA polymerase and sequenced using reverse transcriptase.

Inverse PCR may also be used to amplify or extend the target sequences using divergent primers based on a known region (Triglia T. et al(1988) Nucleic Acids Res 16:8186). The primers may be designed using Oligo 4.0 (National Biosciences Inc, Plymouth Minn.), or another appropriate program, to be 22-30 nucleotides in length, to have a GC content of 50% or more, and to anneal to the target sequence at temperatures about 68°-72° C. The method proposes using several restriction enzymes to generate a suitable fragment in the known region of a gene. The fragment is thereafter circularized by intramolecular ligation and used as a PCR template.

Capture PCR (Lagerstrom M. et al (1991) PCR Methods Applic 1:111-19) is drawn to a method for PCR amplification of DNA fragments adjacent to a known sequence in human and yeast artificial chromosome (YAC) DNA. Capture PCR also requires multiple restriction enzyme digestions and ligations to place an engineered double-stranded sequence into an unknown portion of the DNA molecule before PCR.

Likewise, Parker J. D. et al (1991; Nucleic Acids Res 19:3055-60), teach walking PCR, a method for targeted gene walking which permits retrieval of unknown sequence. PromoterFinder™ a new kit available from Clontech (Palo Alto Calif.) uses PCR, nested primers and PromoterFinder libraries to walk in genomic DNA. This process avoids the need to screen libraries and is useful in finding intron/exon junctions.

Another PCR method, “Improved Method for Obtaining Full Length cDNA Sequences” by Guegler et al, patent application Ser. No. 08/487,112, filed Jun. 7, 1995 and hereby incorporated by reference, employs XL-PCR.™. (Perkin-Elmer) to amplify and/or extend nucleotide sequences.

Preferred libraries for screening for full length cDNAs are ones that have been size-selected to include larger cDNAs. Also, random primed libraries are preferred in that they will contain more sequences which contain the 5′ and upstream regions of genes. A randomly primed library may be particularly useful if an oligo d(T) library does not yield a full-length cDNA. Genomic libraries are useful for extension 5′ of the promoter binding region.

A newer method for analyzing either the size or confirming the nucleotide sequence of sequencing or PCR products is commonly known as “capillary electrophoresis”. Systems for rapid sequencing are available from Perkin Elmer, Beckman Instruments (Fullerton Calif.), and other companies. In general, capillary sequencing employs flowable polymers for electrophoretic separation, four different fluorescent dyes (one for each nucleotide) which are laser activated, and detection of the emitted wavelengths by a charge coupled devise camera. Output/light intensity is converted to electrical signal using appropriate software (eg. Genotyper™ and Sequence Navigator™ from Perkin Elmer) and the entire process from loading of samples to computer analysis and electronic data display is computer controlled. Capillary electrophoresis is particularly suited to the sequencing of small pieces of DNA which might be present in limited amounts in a particular sample. The reproducible sequencing of up to 350 bp of M13 phage DNA in 30 min has been reported (Ruiz-Martinez M. C. et al (1993) Anal Chem 65:2851-8).

Expression of the Nucleotide Sequence:

In accordance with the present invention, α_{1H} SHR polynucleotide sequences which encode α_{1H} SHR, fragments of the polypeptide, fusion proteins or functional equivalents thereof, may be used to generate recombinant DNA molecules that direct the expression of α_{1H} SHR in appropriate host cells. Due to the inherent degeneracy of the genetic code, other DNA sequences which encode substantially the same or a functionally equivalent amino acid sequence, may be used to clone and express α_{1H} SHR. As will be understood by those of skill in the art, it may be advantageous to produce α_{1H} SHR-encoding nucleotide sequences possessing non-naturally occurring codons. Codons preferred by a particular prokaryotic or eukaryotic host (Murray E. et al (1989) Nuc Acids Res 17:477-508) can be selected, for example, to increase the rate of GPG expression or to produce recombinant RNA transcripts having desirable properties, such as a longer half-life, than transcripts produced from naturally occurring sequence.

Also included within the scope of the present invention are polynucleotide sequences that are capable of hybridizing to the nucleotide sequence of SEQ ID NO:1 under conditions of intermediate to maximal stringency. Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.) incorporated herein by reference, and confer a defined “stringency” as explained below.

“Maximum stringency” typically occurs at about Tm-5° C. (5° C. below the Tm of the probe); “high stringency” at about 5° C. to 10° C. below Tm; “intermediate stringency” at about 10° C. to 20° C. below Tm; and “low stringency” at about 20° C. to 25° C. below Tm. As will be understood by those of skill in the art, a maximum stringency hybridization can be used to identify or detect identical polynucleotide sequences while an intermediate (or low) stringency hybridization can be used to identify or detect similar or related polynucleotide sequences. The term “hybridization” as used herein shall include “the process by which a strand of nucleic acid joins with a complementary strand through base pairing” (Coombs J. (1994) Dictionary of Biotechnology, Stockton Press, New York N.Y.) as well as the process of amplification has carried out in polymerase chain reaction technologies as described in Dieffenbach C. W. and G. S. Dveksler (1995, PCR Primer, a Laboratory Manual, Cold Spring Harbor Press, Plainview N.Y.) and incorporated herein by reference.

As used herein a “deletion” is defined as a change in either nucleotide or amino acid sequence in which one or more nucleotides or amino acid residues, respectively, are absent. As used herein an “insertion” or “addition” is that change in a nucleotide or amino acid sequence which has resulted in the addition of one or more nucleotides or amino acid residues, respectively, as compared to the naturally occurring α_{1H} subunit. As used herein “substitution” results from the replacement of one or more nucleotides or amino acids by different nucleotides or amino acids, respectively.

Altered α_{1H} SHR encoding polynucleotide sequences which may be used in accordance with the invention include deletions, insertions or substitutions of different nucleotide residues resulting in a polynucleotide that encodes the same or a functionally/biologically equivalent α_{1H} subunit. The protein may also show deletions, insertions or substitutions of amino acid residues which produce a silent change and result in a functionally equivalent α_{1H} SHR. Deliberate amino acid substitutions may be made on the basis of similarity in polarity, charge, solubility, hydrophobicity, hydrophilicity, and/or the amphipathic nature of the residues as long as the biological activity of an α_{1H} subunit is retained. For example, negatively charged amino acids include aspartic acid and glutamic acid; positively charged amino acids include lysine and arginine; and amino acids with uncharged polar head groups having similar hydrophilicity values include leucine, isoleucine, valine; glycine, alanine; asparagine, glutamine; serine, threonine phenylalanine, and tyrosine.

Also included within the scope of the present invention are alleles of the α_{1H} subunit. As used herein, an “allele” or “allelic sequence” is an alternative form of an α_{1H} subunit, e.g. the α_{1H} SHR isoform. Alleles result from a mutation, i.e., a change in the nucleic acid sequence, and generally produce altered mRNAs or polypeptides whose structure or function may or may not be altered. Any given gene may have none, one or many allelic forms. Common mutational changes which give rise to alleles are generally ascribed to deletions, additions or substitutions of amino acids. Each of these types of changes may occur alone, or in combination with the others, one or more times in a given sequence.

The nucleotide sequences of the present invention may be engineered in order to alter a α_{1H} SHR coding sequence for a variety of reasons, including but not limited to, alterations, which modify the cloning, processing and/or expression of the gene product. For example, mutations may be introduced using techniques which are well known in the art, eg., site-directed mutagenesis to insert new restriction sites, to alter glycosylation patterns, to change codon preference, etc.

Yet another embodiment of the invention proposes ligating a α_{1H} natural, modified or recombinant sequence to a heterologous sequence to encode a fusion protein. For example, for screening of peptide libraries for inhibitors of α_{1H} activity, it may be useful to encode a chimeric α_{1H} SHR protein expressing a heterologous epitope that is recognized by a commercially available antibody. A fusion protein may also be engineered to contain a cleavage site located between a α_{1H} sequence and the heterologous protein sequence, so that the α_{1H} SHR may be cleaved and purified away from the heterologous moiety.

In an alternate embodiment of the invention, the coding sequence of α_{1H} SHR (SEQ ID NO:1) could be synthesized, whole or in part, using chemical methods well known in the art (see Caruthers M. H. et al (1980) Nuc Acids Res Symp Ser 215-23, Horn T. et al(1980) Nuc Acids Res Symp Ser 225-32, etc). Alternatively, the protein itself could be produced using chemical methods to synthesize a α_{1H} SHR amino acid sequence, whole or in part identical to that embodied in SEQ ID NO:2. For example, peptides can be synthesized by solid phase techniques, cleaved from the resin, and purified by preparative high performance liquid chromatography (e.g., Creighton (1983) Proteins Structures And Molecular Principles, W. H. Freeman and Co, New York N.Y.). The composition of the synthetic peptides may be confirmed by amino acid analysis or sequencing (eg, the Edman degradation procedure; Creighton, supra).

Direct peptide synthesis can be performed using various solid-phase techniques (Roberge J. Y. et al (1995) Science 269:202-204) and automated synthesis may be achieved, for example, using the ABI 431A Peptide Synthesizer (Perkin Elmer) in accordance with the instructions provided by the manufacturer. Additionally the amino acid sequence of α_{1H} SHR, or any part thereof, may be altered during direct synthesis and/or combined using chemical methods with sequence(s) from other calcium channel subunits, or any part thereof, to produce a variant polypeptide.

Expression Systems:

In order to express a biologically active α_{1H} SHR of SEQ ID NO:1 including fragments, and biologically equivalent fragments thereof, the nucleotide sequence coding for α_{1H} SHR, or a functional equivalent, is inserted into an appropriate expression vector, i.e., a vector which contains the necessary elements for the transcription and translation of the inserted coding sequence.

Conventional methods, e.g., which are well known to those skilled in the art can be used to construct expression vectors containing a α_{1H} SHR coding sequence and appropriate transcriptional or translational controls. These methods include iii vitro recombinant DNA techniques, synthetic techniques and in vivo recombination or genetic recombination. Such techniques are described in Maniatis et al (1989) Molecular Cloning, A Laboratory Manual, Cold Spring Harbor Press, Plainview N.Y. and Ausubel F. M. et al. (1989) Current Protocols in Molecular Biology, John Wiley & Sons, New York N.Y.

A variety of expression vector/host systems may be utilized to contain and express a α_{1H} SHR coding sequence. These include but are not limited to microorganisms such as bacteria transformed with recombinant bacteriophage, plasmid or cosmid DNA expression vectors; yeast transformed with yeast expression vectors; insect cell systems infected with virus expression vectors (eg, baculovirus); plant cell systems transfected with virus expression vectors (eg, cauliflower mosaic virus, CaMV; tobacco mosaic virus, TMV) or transformed with bacterial expression vectors (eg, Ti or pBR322 plasmid); or animal cell systems.

The “control elements” or “regulatory sequences” of these systems vary in their strength and specificities and are those nontranslated regions of the vector, enhancers, promoters, and 3′ untranslated regions, which interact with host cellular proteins to carry out transcription and translation. Depending on the vector system and host utilized, any number of suitable transcription and translation elements, including constitutive and inducible promoters, may be used. For example, when cloning in bacterial systems, inducible promoters such as the hybrid lacZ promoter of the Bluescript.R™. phagemid (Stratagene, LaJolla Calif.) and ptrp-lac hybrids and the like may be used. The baculovirus polyhedrin promoter may be used in insect cells. Promoters or enhancers derived from the genomes of plant cells (eg, heat shock, RUBISCO; and storage protein genes) or from plant viruses (eg, viral promoters or leader sequences) may be cloned into the vector. In mammalian cell systems, promoters from the mammalian genes or from mammalian viruses are most appropriate. If it is necessary to generate a cell line that contains multiple copies of α_{1H} SHR, vectors based on SV40 or EBV may be used with an appropriate selectable marker.

In bacterial systems, a number of expression vectors may be selected depending upon the use intended for α_{1H} SHR of SEQ ID NO:2 or variant or fragment thereof (collectively referred to as “α_{1H} SHR”. For example, when large quantities of α_{1H} SHR are needed for the induction of antibodies, vectors which direct high level expression of fusion proteins that are readily purified may be desirable. Such vectors include, but are not limited to, the E. coli cloning and expression vector Bluescript.RTM. (Stratagene), in which the α_{1H} SHR coding sequence may be ligated into the vector in frame with sequences for the amino-terminal Met and the subsequent 7 residues of β-galactosidase so that a hybrid protein is produced; pIN vectors (Van Heeke G. & S. M. Schuster (1989) J Biol Chem 264:5503-5509); and the like. pGEX vectors (Promega, Madison Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems are designed to include heparin, thrombin or factor XA protease cleavage sites so that the cloned polypeptide of interest can be released from the α_{1H} SHR moiety at will.

In the yeast Saccharomyces cerevisiae, a number of vectors containing constitutive or inducible promoters such as alpha factor, alcohol oxidase and PGH may be used. For a review of the vectors and promoters, see Ausubel et al (supra).

In cases where plant expression vectors are used, the expression of a α_{1H} SHR coding sequence may be driven by any of a number of promoters. For example, viral promoters such as the 35S or 19S promoters of CaMV (Rhodes C. A. et al (1988) Science 240:204-207) may be used alone or in combination with the omega leader sequence from TMV (Takamatsu N. et al (1987) EMBO J 6:307-311). Alternatively, plant promoters such as the small subunit of RUBISCO (Coruzzi G. et al (1984) EMBO J 3:1671-79; Broglie R. et al (1984) Science 224:838-43); or heat shock promoters (Winter J. and Sinibaldi R. M. (1991) Results Probl Cell Differ 17:85-105) may be used. These constructs can be introduced into plant cells by direct DNA transformation or pathogen-mediated transfection. Refer to Hobbs S or Murry L E in McGraw Yearbook of Science and Technology (1992) McGraw Hill New York N.Y., pp 191-196 for reviews of such techniques.

An alternative expression system which could be used to express α_{1H} SHR encoding sequence is an insect system. In one such system, Autographa californica nuclear polyhedrosis virus (AcNPV) is used as a vector to express foreign genes in Spodoptera frugiperda cells or in Trichoplusia larvae. The α_{1H} SHR coding sequence may be cloned into a nonessential region of the virus, such as the polyhedrin gene, and placed under control of the polyhedrin promoter. Successful insertion of α_{1H} SHR will render the polyhedrin gene inactive and produce recombinant virus lacking coat protein coat. The recombinant viruses are then used to infect S. frugiperda cells or Trichoplusia larvae in which α_{1H} SHR is expressed (Smith G. et al (1983) J Virol 46:584; Engelhard E. K. et al (1994) Proc Nat Acad Sci 91:3224-7).

In mammalian host cells, a number of viral-based expression systems may be utilized. In cases where an adenovirus is used as an expression vector, a α_{1H} SHR coding sequence may be ligated into an adenovirus transcription/translation complex consisting of the late promoter and tripartite leader sequence. Insertion in a nonessential E1 or E3 region of the viral genome will result in a viable virus capable of expressing α_{1H} SHR in infected host cells. (Logan and Shenk (1984) Proc Natl Acad Sci 81:3655-59). In addition, transcription enhancers, such as the rous sarcoma virus (RSV) enhancer, may be used to increase expression in mammalian host cells.

Specific initiation signals may also be required for efficient translation of an inserted α_{1H} SHR sequence. These signals include the ATG initiation codon and adjacent sequences. In cases where α_{1H} SHR, its initiation codon and upstream sequences are inserted into the appropriate expression vector, no additional translational control signals may be needed. However, in cases where only coding sequence, or a portion thereof, is inserted, exogenous transcriptional control signals including the ATG initiation codon must be provided. As well, the initiation codon must be in the correct reading frame to ensure transcription of the entire insert. Exogenous transcriptional elements and initiation codons can be of various origins, both natural and synthetic. The efficiency of expression may be enhanced by the inclusion of enhancers appropriate to the cell system in use (Scharf D. et al (1994) Results Probl Cell Differ 20:125-62; Bittner M. et al (1987) Methods in Enzymol 1 53:51 6-544).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, etc have specific cellular machinery and characteristic mechanisms for such post-translational activities and may be chosen to ensure the correct modification and processing of the introduced, foreign protein.

For long-term, high-yield production of recombinant proteins, stable expression is preferred. For example, cell lines which stably express α_{1H} SHR may be transformed using expression vectors which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection and its presence allows growth and recovery of cells which successfully express the introduced sequences. Resistant clumps of stably transformed cells can be proliferated using tissue culture techniques appropriate to the cell type.

Any number of selection systems may be used to recover transformed cell lines. These include, but are not limited to, the herpes simplex virus thymidine kinase (Wigler M. et al (1977) Cell 11:223-32) and adenine phosphoribosyltransferase (Lowy I. et al (1980) Cell 22:817-23) genes which can be employed in tk.⁻ or aprt⁻ cells, respectively. Also, antimetabolite, antibiotic or herbicide resistance can be used as the basis for selection; for example, dhfr which confers resistance to methotrexate (Wigler M. et al (1980) Proc Natl Acad Sci 77:3567-70); npt, which confers resistance to the aminoglycosides neomycin and G-418 (Colbere-Garapin F. et al (1981) J Mol Biol 150:1-14) and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman S. C. and R. C. Mulligan (1988) Proc Natl Acad Sci 85:8047-51). Recently, the use of visible markers has gained popularity with such markers as anthocyanins, β glucuronidase and its substrate, GUS, and luciferase and its substrate, luciferin, being widely used not only to identify transformants, but also to quantify the amount of transient or stable protein expression attributable to a specific vector system (Rhodes C. A. et al (1995) Methods Mol Biol 55:121-131).

Thus, an aspect of the invention provides recombinant eukaryotic cells that contain the heterologous DNA encoding the a calcium channel subunit of the invention. These are produced by transfection with DNA encoding one or more of the subunits or are injected with RNA transcripts of DNA encoding one or more of the calcium channel subunits. The DNA may be introduced as a linear DNA fragment or may be included in an expression vector for stable or transient expression of the subunit-encoding DNA. Vectors containing DNA encoding human calcium channel subunits of the invention are also provided.

Eukaryotic cells expressing heterologous calcium channels may be used in assays for calcium channel function or, in the case of cells transformed with fewer subunit-encoding nucleic acids than necessary to constitute a functional recombinant human calcium channel, such cells may be used to assess the effects of additional subunits on calcium channel activity. The additional subunits can be provided by subsequently transfecting such a cell with one or more DNA clones or RNA transcripts encoding human calcium channel subunits.

The recombinant eukaryotic cells that express membrane spanning heterologous calcium channels may be used in methods for identifying compounds that modulate calcium channel activity. In particular, the cells are used in assays that identify agonists and antagonists of calcium channel activity in humans and/or assessing the contribution of the various calcium channel subunits to the transport and regulation of transport of calcium ions. Because the cells constitute homogeneous populations of calcium channels, they provide a means to identify agonists or antagonists of calcium channel activity that are specific for each such population.

The recombinant cells of the invention may be used to assess T-type channel function and tissue distribution and to identify compounds that modulate the activity of T-type channels. Because T-type channels are operative in neurons in the thalamus, hypothalamus, and brain stem, and may be involved in autonomic nervous functions, in regulation of cardiovascular activities such as heart rate, arterial and venous smooth muscle innervation and tone, pulmonary rate and other fundamental processes, assays designed to assess such activities and assays to identify modulators of these activities provides a means to understand fundamental physiological processes and also a means to identify new drug candidates for an array of disorders.

As such, the recombinant cells of the invention provide a means to obtain homogeneous populations of calcium channels. Typically, the cells contain the selected calcium channel as the only heterologous ion channel expressed by the cell. Preferably, the α₁ of the calcium channel is one of the disclosed subunits of the invention comprising the amino acid sequences as set forth in one of SEQ ID NOS:1, 3 or 5.

These cells of the invention, which have functional, foreign calcium channels (i.e., functional calcium channels wherein at least one of the α₁-subunit is foreign to the cell) will be useful for, among other purposes, assaying a compound for calcium channel agonist or antagonist activity. First, such a cell can be employed to measure the affinity of such a compound for the functional calcium channel. Secondly, such a cell can be employed to measure electrophysiologically the calcium channel activity in the presence of the compound being tested as well as a ion or molecule, such as Ca++ or Ba++, which is known to be capable of entering the cell through the functional channel. For similar studies which have been carried out with the acetylcholine receptor, see Claudio et al. Science 238 1688-1694 (1987). These methods for assaying a compound for calcium channel agonist or antagonist activity are also contemplated by the present invention.

In another aspect, the recombinant cells of the invention contain heterologous gene(s) (foreign to the cell) with a transcriptional control element, which is active in the cell and responsive to an ion or molecule capable of entering the cell through a functional calcium channel and linked operatively for expression to a structural gene for an indicator protein, can also be employed for assaying a compound for calcium channel agonist or antagonist activity.

The preferred method comprises exposing a culture of such recombinant cells to a solution of a compound being tested for such activity, together with an ion or molecule, which is capable of entering the cells through a functional calcium channel and affecting the activity of the transcriptional control element controlling transcription of the genes for the indicator protein, and comparing the level of expression, in the cells of the culture, of the genes for the indicator protein with the level of such expression in the cells of another, control culture of such cells.

A “control culture,” as clearly understood by the skilled, will be a culture that is treated, in substantially the same manner as the culture exposed to the compound being assayed except that the control culture is not exposed to the compound being assayed. Alternatively, control culture may comprise cells expressing a dysfunctional calcium channel. Levels of expression of the genes for the indicator proteins are ascertained readily by the skilled by known methods, which involve measurements of the concentration of indicator protein via assays for detectable compounds produced in reactions catalyzed by the indicator protein.

As indicated above, indicator proteins are enzymes which are active in the cells of the invention and catalyze production of readily detectable compounds (e.g., chromogens, fluorescent compounds).

In another aspect, the invention provides methods for assaying a compound for calcium channel agonist or antagonist activity employing the recombinant cells of the invention, wherein said cells are exposed to a solution of the compound being tested for such activity. For similar methods applied with Xenopus laevis oocytes and acetylcholine receptors, see Misham et al., Nature, 313, 364 (1985) and, with such oocytes and sodium channels, see Noda et al., Nature 322, 826-828 (1986).

Identification of Transformants Containing the Polynucleotide Sequence:

Although the presence/absence of marker gene expression suggests that the gene of interest is also present, its presence and expression should be confirmed. For example, if the α_{1H} SHR encoding nucleotide sequence is inserted within a marker gene sequence, recombinant cells containing α_{1H} SHR encoding sequences can be identified by the absence of marker gene function. In the alternative, a marker gene can be placed in tandem with a α_{1H} SHR encoding sequence under the control of a single promoter. Expression of the marker gene in response to induction or selection usually indicates expression of α_{1H} SHR as well Alternatively, host cells which contain the coding sequence for α_{1H} SHR and express α_{1H} SHR (SEQ ID NO:2) may be identified by a variety of procedures known to those of skill in the art. These procedures include, but are not limited to, DNA-DNA or DNA-RNA hybridization and protein bioassay or immunoassay techniques which include membrane, solution, or chip based technologies for the detection and/or quantification of the nucleic acid or protein.

The presence of the α_{1H} SHR encoding polynucleotide sequence can be detected by DNA-DNA or DNA-RNA hybridization or amplification using probes, portions or fragments of the α_{1H} SHR nucleotide sequence. Nucleic acid amplification based assays involve the use of oligonucleotides or oligomers based on the α_{1H} SHR sequence to detect transformants containing α_{1H} SHR DNA or RNA. As used herein “oligonucleotides” or “oligomers” refer to a nucleic acid sequence of at least about 10 nucleotides and as many as about 60 nucleotides, preferably about 15 to 30 nucleotides, and more preferably about 20-25 nucleotides which can be used as a probe or amplimer.

The role of α_{1H} SHR in the mobilization of Ca++ as part of the signal transduction pathway can be assayed in vitro. It requires preloading calcium channel expressing cells with a fluorescent dye such as FURA-2 or BCECF (Universal Imaging Corp, Westchester Pa.) whose emission characteristics have been altered by Ca++ binding. When the cells are exposed to one or more activating stimuli artificially or physiologically, Ca++ flux takes place. This flux can be observed and quantified by assaying the cells in a fluorometer or fluorescent activated cell sorter. The measurement of Ca++ mobilization in mobilization assays is well known. Briefly, in a calcium mobilization assay, cells expressing the target receptor are loaded with a fluorescent dye that chelates calcium ions, such as FRRA-2. Upon addition of a calcium channel modulator to the cells expressing a calcium channel, the target modulator binds to the calcium channel and calcium is released from the intracellular stores. The dye chelates these calcium ions. Spectrophotometric determination of the ratio for dye:calcium complexes to free dye determine the changes in intracellular calcium concentrations upon addition of the target modulator. Hits from screens and other test compounds can be similarly tested in this assay to functionally characterize them as agonists or antagonists. Increases in intracellular calcium concentrations are expected for compounds with agonist activity while compounds with antagonist activity are expected to block target modulator stimulated increases in intracellular calcium concentrations. See U.S. Pat. No. 6,420,137 and similar patents.

In preferred embodiments, the cells express such heterologous calcium channel subunits and include one or more of the subunits in membrane-spanning heterologous calcium channels. In more preferred embodiments, the eukaryotic cells express functional, heterologous calcium channels that are capable of gating the passage of calcium channel-selective ions and/or binding compounds that, at physiological concentrations, modulate the activity of the heterologous calcium channel. In certain embodiments, the heterologous calcium channels include at least one heterologous calcium channel subunit. In most preferred embodiments, the calcium channels that are expressed on the surface of the eukaryotic cells are composed substantially or entirely of subunits encoded by the heterologous DNA or RNA. In preferred embodiments, the heterologous calcium channels of such cells are distinguishable from any endogenous calcium channels of the host cell.

A variety of protocols for detecting and measuring the expression of α_{1H} SHR, using either polyclonal or monoclonal antibodies specific for the protein are known in the art. Examples include enzyme-linked immunosorbent assay (ELISA), radioimmunoassay (RIA) and fluorescent activated cell sorting (FACS). A two-site, monoclonal-based immunoassay utilizing monoclonal antibodies reactive to two non-interfering epitopes on α_{1H} SHR is preferred, but a competitive binding assay may be employed. These and other assays are described, among other places, in Hampton R. et al (1990, Serological Methods, a Laboratory Manual, APS Press, St. Paul Minn.) and Maddox D. E. et al (1983, J Exp Med 158:1211).

A wide variety of labels and conjugation techniques are known by those skilled in the art and can be used in various nucleic and amino acid assays. Means for producing labelled hybridization or PCR probes for detecting sequences related to α_{1H} SHR include oligolabelling, nick translation, end-labelling or PCR amplification using a labelled nucleotide. Alternatively, the α_{1H} SHR sequence, or any portion of it, may be cloned into a vector for the production of an mRNA probe. Such vectors are known in the art, are commercially available, and may be used to synthesize RNA probes in vitro by addition of an appropriate RNA polymerase such as T7, T3 or SP6 and labelled nucleotides.

A number of companies such as Pharmacia Biotech (Piscataway N.J.), Promega (Madison Wis.), and US Biochemical Corp (Cleveland Ohio) supply commercial kits and protocols for these procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. Nos. 3,817,837; 3,850,752; 3,939,350; 3,996,345; 4,277,437; 4,275,149 and 4,366,241. Also, recombinant immunoglobulins may be produced as shown in U.S. Pat. No. 4,816,567 incorporated herein by reference.

Purified α_{1H} SHR Polypeptides:

Host cells transformed with a α_{1H} SHR encoding nucleotide sequence may be cultured under conditions suitable for the expression and recovery of the encoded protein from cell culture. The protein produced by a recombinant cell may be secreted or may be contained intracellularly depending on the sequence and/or the vector used. As will be understood by those of skill in the art, expression vectors containing α_{1H} SHR can be designed with signal sequences which direct secretion of α_{1H} SHR through a particular prokaryotic or eukaryotic cell membrane. Other recombinant constructions may join α_{1H} SHR to nucleotide sequence encoding a polypeptide domain which will facilitate purification of soluble proteins (Kroll D. J. et al (1993) DNA Cell Biol 12:441-53; see also above discussion of vectors containing fusion proteins).

An α_{1H} SHR subunit may also be expressed as a recombinant protein with one or more additional polypeptide domains added to facilitate protein purification. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp, Seattle Wash). The inclusion of a cleavable linker sequences such as Factor XA or enterokinase (Invitrogen, San Diego Calif.) between the purification domain and GPG is useful to facilitate purification.

Proposed Uses of the Various α_{1H} Subunits of the Invention:

The rationale for diagnostic and potential therapeutic uses of the herein disclosed α_{1H} subunit sequences is based on the nucleotide and amino acid sequences, their homology to the human α_{1H} protein, their tissue distribution in (Provide details) and the known associations and functions of said proteins. The nucleic acid sequence presented in SEQ ID NO:1, its complement, fragments or oligomers, and anti-α_{1H} antibodies may be used as diagnostic compositions in assays of cells, tissues or their extracts. Purified α_{1H} SHR encoding nucleic acid molecule or polypeptide can be used as the positive controls in their respective nucleic acid or protein based assays for conditions or diseases characterized by the excess expression or aberrant expression or activity of native T-type calcium channel α_{1H} subunit. Antisense molecules, antagonists or inhibitors capable of specifically binding the α_{1H} encoding nucleic acid molecule or the encoded polypeptide can be used as pharmaceutical compositions for conditions or diseases characterized by the aberrant expression of a T-type α_{1H} calcium channel subunit.

Furthermore, calcium influx via low-voltage-gated calcium channels and intracellular calcium signaling plays a role in hormone secretion, cardiac pacing and disorders of the CNS. Thus, it is contemplated that the present invention will find use in investigations regarding the inactivation of low-voltage gated calcium channel subunits such as the α_{1H} subunit by any of several means (e.g., in investigations pertaining to such areas as cancer pathogenesis, cardiac arrhythmias etc.)

The prior art is replete with teachings suggesting that the T-type calcium channel α_{1H} subunit may be involved in the origin of cancers (e.g., lung cancer, breast cancer, etc. Indeed, interest in the physiological roles of Ca++ channels has increased, due to finding that mutations in these genes can lead to human diseases. In addition to potential role(s) in cardiac and CNS pathogenesis and pathologies involving the circadian rhythm, defects in the auxiliary subunits of Ca++ channels have been described in non-human models of absence epilepsy. These include mouse strains that have lost the expression of the beta auxiliary and the recently discovered .gamma subunit. See Letts et al., Nat. Genet., 19:340-347, 1998; and Burgess et al., Cell 88:385-392,1997. Thus, it is contemplated that the present invention will find use in the development of methods to identify and test for the presence of inherited defects in T-type calcium channel subunits in other species, including humans. It is also contemplated that the present invention will find use in assessing calcium channel defects associated with epileptic and other pathological phenotypes.

Clinical Applications

In relation to therapeutic treatment of various disease states, the availability of DNA encoding a murine calcium channel subunits permits identification of any alterations in such genes (e.g., mutations) which may correlate with the occurrence of certain disease states. Thus, in one aspect, the herein disclosed sequences may be used as a probe to identify substantially similar genes in other species, preferably human. In addition, the creation of animal models of such disease states becomes possible, by specifically introducing such mutations into synthetic DNA fragments that can then be introduced into laboratory animals or in vitro assay systems to determine the effects thereof.

In another broad aspect, genetic screening can be carried out using the nucleotide sequences as probes. Thus, nucleic acid samples from subjects having pathological conditions suspected of involving alteration/modification of any one or more of the calcium channel subunits can be screened with appropriate probes to determine if any abnormalities exist with respect to any of the endogenous calcium channels. Similarly, subjects having a family history of disease states related to calcium channel dysfunction can be screened to determine if they are also predisposed to such disease states.

It is well known that mutations that lead to over expression , e.g., enhanced expression of channels or that reduce inactivation might help tip the balance to overexcitability. Indeed, enhanced expression of T-type channels have been detected in various animal models of for example, epilepsy, cardiac h hypertrophy and heart failure. As well, enhanced expression has also been observed in neuronal injury. See Edward Perez-Reyes, Molecular Physiology of Low-Voltage-Activated T-type Calcium Channels, Physiol. Rev., 83:117-161, 2003, incorporated in its entirety by reference herein. Consequently, the sequences of the invention may be used to probe a biological specimen and identify a variant sequence whose expression may be correlated to a diseased phenotype. For example, antibodies specific f for a sequence of the invention may be used to identify a T-type α_{1H} calcium channel variant in a biological sample, and the sequence of the so identified variant may thereafter be compared to a reference sequence and mutations, if any identified. The mutated sequence, in turn, may then be used to correlate a disease status with its expression.

The regulation of the T-type calcium channel α_{1H} subunit expression provides an opportunity for early intervention in conditions based on aberrant expression or a dysfunctional α_{1H} subunit relative to normal.

In an analogous manner, appropriate delivery of vectors expressing antisense sequences, peptide nucleic acids (PNA), or inhibitors of α_{1H} subunit can be used to prevent or treat excessive or inadequate calcium mobilization resulting from a dysfunctional α_{1H} subunit resulting in damage to neuronal or cardiac tissue. Delivery of these therapies, as noted below, will necessarily be tissue/cell specific and depend on the diagnosis, size and status of the diaseas/damage.

The regulation of calcium flux or α_{1H} subunit expression provides an opportunity to intervene in various disorders involving a dysfunctional T-type calcium channel. Inappropriate activation or aberrant expression or activation of a T-type calcium channel may result in the tissue damage and destruction seen in cardiac or neuronal disease states For example, transfection of the cardiac cells expressing a dysfunctional T-type calcium channel subunit, for example, with vectors expressing antisense sequences or with liposomes bearing PNAs or inhibitors of human α_{1H} subunit can be used to treat or correct a dysfunctional calcium channel and subsequent correction of the underlying disease state resulting from the dysfunctional calcium channel or excessive or inadequate calcium flux.

GPG Antibodies:

The prior art is replete with information pertaining to the the production of antibodies. Such information can be used to produce antibodies to the α_{1H} subunit of SEQ ID NO:2. Such antibodies include, but are not limited to, polyclonal, monoclonal, chimeric, single chain, Fab fragments and fragments produced by a Fab expression library. Neutralizing antibodies, ie, those which inhibit dimer formation, are especially preferred for diagnostics and therapeutics.

For the production of antibodies, various hosts including goats, rabbits, rats, mice, etc may be immunized by injection with the sequence encoded by SEQ ID NO:1 or the encoded protein of SEQ ID NO:2, or any portion, fragment or oligopeptide which retains immunogenic properties. Depending on the host species, various adjuvants may be used to increase immunological response. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful human adjuvants.

Monoclonal antibodies to SEQ ID NO:2 or a variant, biologically active fragment or derivative thereof may be prepared using any technique which provides for the production of antibody molecules by continuous cell lines in culture. These include but are not limited to the hybridoma technique originally described by Koehler and Milstein (1975 Nature 25 256:495-497), the human B-cell hybridoma technique (Kosbor et al (1983) Immunol Today 4:72; Cote et al (1983) Proc Natl Acad Sci 80:2026-2030) and the EBV-hybridoma technique (Cole et al (1985) Monoclonal Antibodies and Cancer Therapy, Alan R. Liss Inc, New York N.Y., pp 77-96). As well, techniques developed for the production of “chimeric antibodies”, the splicing of mouse antibody genes to human antibody genes to obtain a molecule with appropriate antigen specificity and biological activity can be used (Morrison et al (1984) Proc Natl Acad Sci 81:6851-6855; Neuberger et al (1984) Nature 312:604-608; Takeda et al (1985) Nature 314:452-454). Alternative techniques for the production of single chain antibodies (U.S. Pat. No. 4,946,778) may also be adapted to produce anti-α_{1H} SHR (SEQ ID NO:2 ) specific single chain antibodies.

Antibodies may also be produced by inducing in vivo production in the lymphocyte population or by screening recombinant immunoglobulin libraries or panels of highly specific binding reagents as disclosed in Orlandi et al (1989, Proc Natl Acad Sci 86: 3833-3837), and Winter G and Milstein C. (1991; Nature 349:293-299).

Antibody fragments which contain specific binding sites for an α_{1H} subunit may also be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. On the other hand, Fab expression libraries may be constructed to allow rapid and easy identification of monoclonal Fab fragments with the desired specificity (Huse W. D. et al (1989) Science 256:1275-1281).

α_{1H} subunit -specific antibodies are useful for the diagnosis of conditions and diseases associated with excessive expression of α_{1H} subunit. A variety of protocols for competitive binding or immunoradiometric assays using either polyclonal or monoclonal antibodies with established specificities are well known in the art. Such immunoassays typically propose forming complexes between α_{1H} polypeptide and its specific antibody and the measurement of complex formation. A two-site, monoclonal-based immunoassay utilizing monoclonal antibodies reactive to two noninterfering epitopes on a specific α_{1H} protein is preferred, but a competitive binding assay may also be employed. These assays are well known to one skilled in the art. See, for example, Maddox D. E. et al (1983, J Exp Med 158:1211).

Diagnostic Assays Using α_{1H} Subunit Specific Antibodies of the Invention:

Particular α_{1H} subunit-specific antibodies will find use in the diagnosis of conditions or diseases characterized by excessive or inadequate, e.g., aberrant expression of an α_{1H} subunit. Diagnostic assays for aberrant α_{1H} subunit expression or activity include methods utilizing the antibody and a label to detect α_{1H} subunit in a subject's body fluids, cells, tissues or extracts of such tissues. The polypeptides and antibodies of the present invention may be used with or without modification. Frequently, the polypeptides and antibodies will be labeled by joining them, either covalently or noncovalently, with a reporter molecule. A wide variety of reporter molecules are known, several of which were described above.

A variety of protocols for measuring α_{1H} subunit expression or activity level using either polyclonal or monoclonal antibodies specific for the respective protein are known in the art. Examples include enzyme-linked immunosorbent assay (ELISA), radioimmunoassay (RIA) and fluorescent activated cell sorting (FACS). A two-site, monoclonal-based immunoassay utilizing monoclonal antibodies reactive to two non-interfering epitopes on an α_{1H} subunit is preferred, but a competitive binding assay may be employed. These assays are described, among other places, in Maddox, D. E. et al (1983, J Exp Med 158:1211).

To be accurate and in order to provide a basis for the diagnosis of disease, normal or standard values for the respective α_{1H} subunit expression or activity level must be established. This is accomplished by combining body fluids or cell extracts taken from normal subjects, either animal or human, with antibody to the respective α_{1H} subunit under conditions suitable for complex formation which are well known in the art. The amount of standard complex formation may be quantified by comparing it with a dilution series of positive controls where a known amount of antibody is combined with known concentrations of purified α_{1H} subunit. Thereafter, standard values obtained from normal samples may be compared with values obtained from samples from subjects potentially affected by a disorder or disease related to aberrant α_{1H} subunit expression. Deviation between standard and subject values, in turn, establishes the presence of disease state.

Uses of the Nucleic Acid Molecule Encoding an α_{1H} Subunit:

A nucleic acid, α_{1H} subunit encoding sequence, or any part thereof, may be used for diagnostic and/or therapeutic purposes. For diagnostic purposes, the nucleic acid molecules of the invention, e.g., SEQ ID NO:1 or its variant or fragment thereof, may be used to detect and quantitate gene expression in conditions or diseases characterized or mediated by a dysfunctional T-type calcium channel α_{1H} subunit. These specifically include, but are not limited to cardiovascular pathologies such as angina, vascular, such as hypertension, and urologic, hepatic, reproductive, adjunctive therapies for reestablishing normal heart rate and cardiac output following traumatic injury, heart attack and other cardiac injuries; treatments of myocardial infarct (MI), post-MI and in an acute setting, neuronal pathologies of the central nervous system etc. Included in the scope of the invention are oligonucleotide sequences, antisense RNA and DNA molecules, PNAs and ribozymes, which function to inhibit translation of an α_{1H} subunit.

Another aspect of the subject invention is to provide for hybridization or PCR probes which are capable of detecting polynucleotide sequences, including genomic sequences, encoding α_{1H} subunit or closely related molecules. The specificity of the probe, whether it is made from a highly conserved region, eg, 10 unique nucleotides in the 5′ regulatory region, or a less conserved region, e.g., between cysteine residues especially in the 3′ region, and the; stringency of the hybridization or amplification (high, intermediate or low) will determine whether the probe identifies only naturally occurring α_{1H} subunit or related sequences. Mutated sequences may also be detected in like manner.

Therapeutics

An antisense sequence based on the α_{1H} subunit sequence of this application may be useful in the treatment of various conditions or diseases. By introducing antisense sequence into cells, gene therapy can be used to treat conditions or diseases characterized by a dysfunctional T-type calcium channel α_{1H} subunit. In such instances, the antisense sequence binds with the complementary DNA strand and either prevents transcription or stops transcript elongation (Hardman J. G. et al. (1996) Goodman and Gilson's The Pharmacological Basis of Therapeutics. McGraw Hill, New York N.Y.).

Expression vectors derived retroviruses, adenovirus, herpes or vaccinia viruses, or from various bacterial plasmids, may be used for delivery of antisense sequences to the targeted cell population. Methods which are well known to those skilled in the art can be used to construct recombinant vectors which will express the antisense sequence. See, for example, the techniques described in Maniatis et al (supra) and Ausubel et al (supra). Alternatively, antisense molecules such as PNAs can be produced and delivered to target cells or tissues in liposomes.

Alternatively, the full length cDNA sequence and/or its regulatory elements of the α_{1H} subunit, e.g., SEQ ID NO:2 will enable researchers to use α_{1H} subunit as a tool in sense (Youssoufian H. and H. F. Lodish 1993 Mol Cell Biol 13:98-104) or antisense (Eguchi et al (1991) Annu Rev Biochem 60:631-652) investigations or regulation of gene function. Such technology is now well known in the art, and sense or antisense oligomers, or larger fragments, can be designed from various locations along the coding or control regions.

Detection and Mapping of Related Polynucleotide Sequences:

The nucleic acid sequences of the invention can also be used to generate hybridization probes for mapping the naturally occurring genomic sequence corresponding to the α_{1H} subunit in other species such as humans. The sequence may be mapped to a particular chromosome or to a specific region of the chromosome using well known techniques. These include in situ hybridization to chromosomal spreads, flow-sorted chromosomal preparations, or artificial chromosome constructions such as YACs, bacterial artificial chromosomes (BACs), bacterial P1 constructions or single chromosome cDNA libraries (reviewed in Price C. M. (1993) Blood Rev 7:127-34 and Trask B. J. (1991) Trends Genet 7:149-54).

In situ hybridization of chromosomal preparations and physical mapping techniques such as linkage analysis using established chromosomal markers are invaluable in extending genetic maps. Examples of genetic maps can be found in Science (1995; 270:410f and 1994; 265:1981f). Often the placement of a gene on the chromosome of another mammalian species may reveal associated markers even if the number or arm of a particular human chromosome is not known. New sequences can be assigned to chromosomal arms, or parts thereof, by physical mapping. This will provide valuable information to investigators searching for disease genes using positional cloning or other gene discovery techniques. Once a disease or syndrome is crudely localized by genetic linkage to a particular genomic region, any sequences mapping to that area may represent associated or regulatory genes for further investigation. The nucleotide sequence of the subject invention may also be used to detect differences in the chromosomal location due to translocation, inversion, etc. between normal, carrier or affected individuals.

Pharmaceutical Compositions:

The present invention comprises pharmaceutical compositions which may comprise antibodies, antagonists, or inhibitors of a α_{1H} subunit, alone or in combination with at least one other agent, such as stabilizing compound, which may be administered in any sterile, biocompatible pharmaceutical carrier, including, but not limited to, saline, buffered saline, dextrose, and water.

Antagonists, or inhibitors of α_{1H} subunit can be administered to a patient alone, or in combination with other agents, drugs or hormones, in pharmaceutical compositions where it is mixed with excipient(s) or pharmaceutically acceptable carriers. In one embodiment of the present invention, the pharmaceutically acceptable carrier is pharmaceutically inert.

Further details on techniques for formulation and administration may be found in the latest edition of “Remington's Pharmaceutical Sciences” (Mack Publishing Co, Easton Pa.). Although local delivery is desirable, there are other means, for example, oral; parenteral delivery, including intra-arterial (directly to the tumor), intramuscular, subcutaneous, intramedullary, intrathecal, intraventricular, intravenous, intraperitoneal, or intranasal administration.

The examples below are provided to illustrate the subject invention, These examples are provided by way of illustration and are not included for the purpose of limiting the invention.

### EXAMPLE 1

Cloning of Rat Alpha 1H T-type Channels

Sprague-Dawley rat adrenal total RNA was purchased from Clontech. Adrenal glands were dissected from SHR and WKY rats and RNA isolated by Trizol (Invitrogen) extraction method. Complimentary DNA was synthesized and used as template in PCR reactions. Primary and nested PCR reactions used various combinations of the following forward and reverse oligonucleotide primers and amplified either full- or partial length fragments of alpha1h cDNA:

Forward:
GCTCCCaagcttactagtCCCAGTGACAGCGCCGCCCGGACTATG
GCGCCGaagcttactagtCCACGGGGACGCCGCTAGCCACC
CTAGCCaagcttactagtTGCTGCCCTCCGCCACCATGACCG
AGCGAGaagcttactagtGCCACCATGACCGAGGGCACGCTGG
AACAGGaagcttactagtTGTGCGCCACCCTCGCCGCCATCC
ACTCTGaagcttactagtGTATCTACCATGCTGACTGCCACGTGGAGGGG
C
Reverse:
GGCTGCctcgagCCTCTAGGTGCCCGTTAGGGGTCACTGCCA
GGGGTTctcgagCTGCACGGGCTGCTGGTCGATGCCCAC
GCGAATctcgagAGCGGCGAGTGTGTGAATAGTCTGCGTAGTAGGGCC
GTCATGctcgagAGACGGGATGTCTGCTGCCTCTCCTGGGAT
AGGAATctcgagTCCTTCCCAGGACACAGCCTCTCCTCCTGA

Amplified cDNA fragments were subcloned into either pBluescript or pCR-XL-TOPO plasmids. DNA was prepared from transformed bacteria and sequenced by standard methods. Nucleotide and predicted amino acid sequences were compared to each other and available rat alpha 1H GenBank entries.

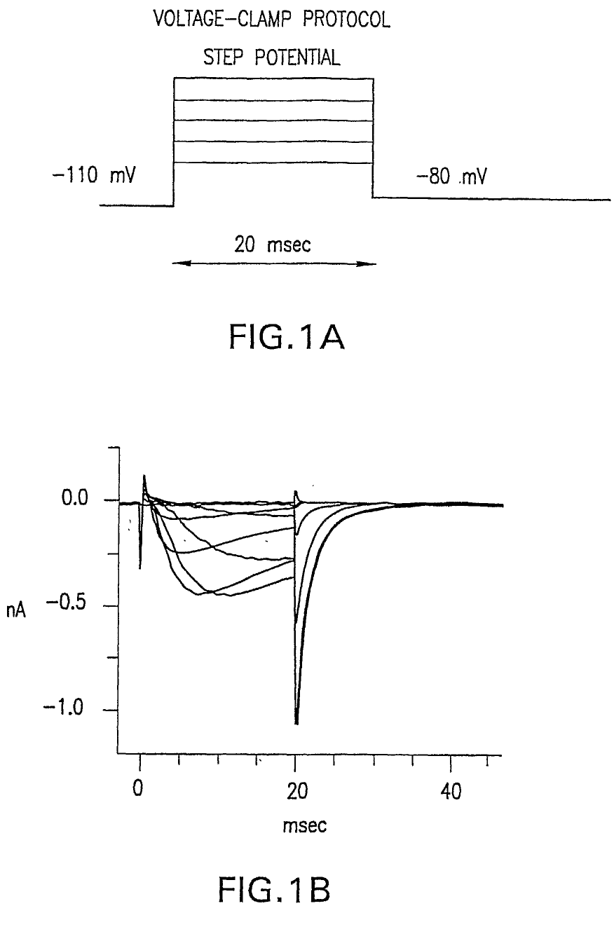

Cloned fragments encoding the consensus amino acid sequence were assembled by standard restriction enzyme digestion and ligation. This assembled clone was then transferred to pcDNA3.1 for transient expression in mammalian cells. Functional data is shown in FIG. 1 for the SHR channel..

### SUMMARY OF SEQUENCES

SEQ ID NO:1 Nucleotide sequence of the α_{1H} subunit designated herein as α_{1H} SHR subunit.

SEQ ID NO:2 Deduced amino acid sequence of the α_{1H} SHR subunit

SEQ ID NO:3 Nucleotide sequence of the α_{1H} subunit designated herein as α_{1H} WKY subunit.

SEQ ID NO:4 Deduced amino acid sequence of the α_{1H} WKY subunit.

SEQ ID NO:5 Nucleotide sequence of the α_{1H} subunit designated herein as α_{1H} S-D subunit.

SEQ ID NO:6 Deduced amino acid sequence of the α_{1H} S-D subunit

1. An isolated nucleic acid molecule comprising a sequence of nucleotides encoding a murine T-type calcium channel α_{1H} subunit selected from the group consisting of:
(a) a sequence of nucleotides that encodes a murine T-type calcium channel α_{1H} subunit and comprises the sequence of nucleotides set forth in one of SEQ ID NOS:1 or 5;
(b) a sequence of nucleotides having at least 95% sequence identity or is exactly complementary to the nucleotide sequence set forth in SEQ ID NO:1 or 5, and
(c) a nucleotide sequence varying from the nucleotide sequence specified in (a) or (b) as a result of degeneracy of the genetic code.

2. A substantially pure polypeptide comprising an amino acid sequence selected from the group consisting of: (i) an amino acid sequence coded by the isolated nucleic acid molecule of claim 1; (ii) homologues of the amino acid sequences of (i) in which one or more amino acids has been added, deleted, replaced or chemically modified in the region, or adjacent to the region, where the amino acid sequences differs from the original amino acid sequence, coded SEQ ID NOS: 1 or 5.

3. A substantially pure polypeptide comprising an amino acid sequence encoded by the nucleotide sequence as set forth in one of SEQ ID NOS: 1 or 5.

4. A substantially pure polypeptide comprising an amino acid sequence as set forth in one of SEQ ID NOS: 2 or 6.

5. An expression vector comprising the nucleic acid molecule of claim 1 operably linked to a regulatory nucleotide sequence that controls expression of the nucleic acid molecule in a suitable host cell.

6. A recombinant host cell transfected by the expression vector of claim 5.

7. A method for detecting the presence of a nucleic acid sequence of α_{1H} in a biological sample, comprising the steps of: (a) hybridizing to nucleic acid material in said biological sample the nucleic acid molecule of claim 1 under conditions favoring the formation of a hybridization complex; and (b) detecting said hybridization complex; wherein the presence of said hybridization complex correlates with the presence of an variant nucleic acid sequence in the said biological sample.

8. A method for determining the level of a nucleic acid sequences of α_{1H} subunit or a variant thereof in a biological sample comprising the steps of: (a) hybridizing to nucleic acid material of said biological sample the nucleic acid sequences of claim 1; and (b) determining the amount of hybridization complexes and normalizing said amount to provide the level of the α_{1H} subunit or variant thereof encoding nucleic acid sequences in the sample.

9. A method for detecting the level of the polypeptide variant of claim 4 or a biologically active fragment or variant thereof in a biological sample, comprising the steps of: (a) contacting said biological sample with a detectable antibody having binding specificity for a polypeptide of SEQ ID NO: 2 or 6, thereby forming an antibody-polypeptide complex; and (b) detecting the amount of said antibody-polypeptide complex and normalizing said amount to provide the level of said amino acid sequence in the sample.

10. A method for identifying lead compounds for a pharmacological agent useful in the treatment of disease associated with increased or decreased voltage regulated calcium influx mediated by a rat T-type calcium channel comprising:
(i) providing a cell expressing a rat T-type calcium channel subunit polypeptide designated herein as α_{1H}; said calcium channel subunit comprising the amino acid sequence as set forth in one of SEQ ID NOS: 2, 4 or 6;
(ii) contacting the cell with a candidate pharmacological agent under conditions which, in the absence of the candidate pharmacological agent, to thereby cause a first amount of voltage regulated calcium influx into the cell; and
(iii) determining a test amount of voltage regulated calcium influx as a measure of the effect of the lead compounds for a pharmacological agent on the voltage regulated calcium influx mediated by a human T-type calcium channel, wherein (a) the test amount of voltage regulated calcium influx which is less than the first amount indicates that the candidate pharmacological agent is a lead compound for a pharmacological agent which reduces voltage regulated calcium influx and (b) wherein a test amount of voltage regulated calcium influx which is greater than the first amount indicates that the candidate pharmacological agent is a lead compound for a pharmacological agent which increases voltage regulated calcium influx.

11. The method of claim 10, further comprising loading said cell with a calcium-sensitive dye which is detectable in the presence of calcium, wherein the calcium-sensitive dye is detected as a measure of the voltage regulated calcium influx.

12. A method for identifying compounds which selectively bind a T-type calcium channel α_{1H} subunit comprising, (i) providing a test cell preparation, wherein said cell expresses a polypeptide encoded by the nucleic acid molecule of claim 1, (ii) providing a control cell preparation, wherein said cell expresses a rat T-type calcium channel non-α_{1H} subunit, with the proviso that the cell in the control cell preparation is identical to the test cell except for the expression of a non-α_{1H} being expressed, (iii) contacting the test cell preparation and the control cell preparation with a compound, and (iv) determining the binding of the compound to the test cell preparation and the control cell preparation, wherein a compound which binds the test cell preparation but does not bind the control cell preparation is a compound which selectively binds the a mammalian T-type calcium channel α_{1H} subunit.

13. A diagnostic method for predicting an oncogenic potential of a sample of cells, comprising:
(a) determining, in the sample levels of expression of a target gene sequence as claimed in claim 8 and comparing said sequence with the sequence as set forth in GenBank Accession No. AF2902 13 to determine mutations in the target sequences or its complement, wherein excessive or insufficient levels of expression of said target sequence relative to normal is predictive of the oncogenic potential of said cells.

14. The nucleic acid molecule of claim 1, wherein said nucleic acid molecule is cDNA.

15. A method of producing the recombinant protein according to claim 3, comprising:
(a) inserting the nucleic acid sequence as set forth in SEQ ID NO: 1 or 5 or a fragment or variant thereof into an expression vector;
(b) transferring the expression vector into a host cell; or transfecting or transforming a host cell with the expression vector of step (a) above;
(c) culturing the host organism under conditions appropriate for amplification of the vector and expression of the protein; and
(d) harvesting the recombinant protein from the culture.

16. A method for identifying compounds that modulate the activity of the polypeptide of claim 3, the method comprising:
comparing the difference in the amount of transcription of a reporter gene in a cell in the presence of the compound with the amount of transcription in the absence of the compound, or with the amount of transcription in the absence of a heterologous T-type calcium channel α_{1H} subunit, whereby compounds that modulate the activity of the heterologous calcium channel subunit in the cell are identified, wherein the cell comprises a nucleic acid molecule that encodes a reporter gene construct containing a reporter gene in operative linkage with one or more transcription control elements that is regulated by a calcium channel and furthermore the cell is a eukaryotic cell transfected with a nucleic acid molecule comprising the coding portion of the sequence of nucleotides set forth in one of SEQ ID NO: 1 or 5.

17. A method for identifying a test compound capable of modulating the activity of the polypeptide of claim 3, the method comprising:
(i) suspending a eukaryotic cell in a solution containing the compound and a calcium channel selective ion;
(ii) depolarizing the cell membrane of the cell, and
(iii) detecting the current or ions flowing into the cell,
wherein the eukaryotic cell comprises a functional calcium channel that contains at least one subunit encoded by a heterologous nucleic acid comprising the coding portion of the sequence of nucleotides set forth in SEQ ID NOs: 1 or 5, and
wherein the current that is detected is different from that produced by depolarizing the same or a substantially identical cell in the presence of the same calcium channel selective ion but in the absence of the test compound.

18. The method of claim 17, wherein prior to the depolarization step the cell is maintained at a holding potential which substantially inactivates calcium channels that are endogenous to the cell.

19. A method for determining whether a test compound inhibits calcium channel activity in cells, said method comprising:
(a) culturing recombinant cells expressing a functional calcium channel including as a component a functional polypeptide according to claim 3 under conditions where intracellular calcium concentrations depend on calcium channel activity; and
(b) measuring intracellular calcium concentrations in the cultured recombinant cells in the presence and absence of the test compound to determine whether the intracellular calcium concentration in the recombinant cells in the presence of the test compound is lower than the intracellular calcium concentration in the cells cultured in the absence of the test compound, wherein a test compound which lowers said calcium concentration is considered to be a calcium channel inhibitor.

20. A method as in claim 19, wherein intracellular calcium concentration is measured by observing a change in fluorescence of a calcium sensitive dye which is introduced to the cultured recombinant cells prior to the test compound.


SEQUENCE LISTING (6 sequences)

SEQ ID NO: 1 - DNA, 7426 bp, Rat
ccacggggacgccgctagccaccggagcgaggtgctgccctccgccaccatgaccgagggcacgctggcagcggacgaagtccgggtgcccctgggcgcttcgccgccggcccctgcagcgccggtgagggcttccccagcgagccctggggcgccggggcgcgaggagcagggaggatccgggtcgggcgtgttggctcccgagagcccagggaccgagtgtggtgcggacctgggcgccgacgaggaacagccggtcccatacccagctctggctgccacagtcttcttctgcctcgggcaaaccacgcggccgcgcagctggtgcctccgactggtttgtaacccgtggttcgagcacatcagcatgctggtcatcatgctgaactgcgtgacactgggcatgttcaggccctgtgaggatgttgagtgccgctccgaacgttgcagcatcttggaggccttcgacgacttcatctttgccttcttcgccgtggagatggtgatcaagatggtggctttggggctgtttgggcaaaaatgctacctgggtgacacctggaacaggctggacttcttcattgtcatggcgggcatgatggagtactctctggacggacacaacgtgagcctctctgccatccgaaccgtgcgtgtgctgcggcccctccgcgccatcaaccgagtccccagtatgcggatcctggtcactctgctgctggacacgctgcccatgcttgggaatgtcctcctcctctgcttcttcgtcttcttcatcttcggcattgttggggtccagctctgggctggcctgcttcggaaccgatgcttcctggacagcgccttcgtcaggaacaacaacctgaccttcttgcggccatactaccagacggaggagggtgaggagaaccctttcatctgctcctcccgccgtgacaacggcatgcagaagtgctcgcacatccccagccgccgtgagcttcgagtgcagtgcacactcggctgggaggcctatgggcagccacaggctgaggatgggggtgctggccgcaacgcctgtatcaactggaaccagtattacaacgtgtgccgctcgggggaattcaaccctcacaacggtgccatcaacttcgacaacattggctacgcttggattgccatcttccaggtcatcacactggagggctgggtggacatcatgtactacgtcatggatgcccactcgttctacaacttcatctacttcatcctcctcatcattgtgggctccttcttcatgatcaacctgtgcctggtggtgatagccacacagttctcagagacaaagcaaagggaaaaccagctgatgcgagaacagcgggcccgctatctgtccaacgacagcactctggccagcttctcagagcccggcagctgctacgaggagctcctcaagtatgtaggccacatcttccggaaggttaaacgccgtagcctgcgtctttatgcccgctggcagagccgctggcgtaagaaggtggatcccagcagtaccgtgcatggccaaggccctgggcggcggccacgacgggcaggcaggcgtacagcttcagtgcaccatctggtctaccaccaccaccaccaccatcaccaccattaccactttagccacggtggcccacgcaggcccagcccagagccaggtgctggtgacaacaggttggtcagggcctgtgcgccaccctcgccgccatccccaggccatgggccaccagactctgagtctgtgcacagtatctaccatgctgactgccacgtggaggggccgcaggaacgagcccgagtggcacactccatagccactgctgctagcctcaagctggcctcaggtttgggtaccatgaactaccccaccatcctaccttcaggaacagtcaacagcaaaggtggcaccagctcacgacccaaggggctacgaggtgctggcgccccaggggctgcagtacacagccctctgagcctgggaagccccagaccctatgagaagatccagcatgtggtgggagaacaaggactaggccgagcctctagccacctgtcaggcctgagtgtgccttgccccctgcccagcccccaggctggcacgctgacctgtgagctgaagagctgcccatattgtgccagcgccctggaggaccccgagtttgaattcagtggctcagagagcggggactcggatgcccacggagtctatgagtttacccaggatgtacggcatggggattgtcgggaccctgtgcagcagccccatgaagtgggcacaccaggccacagcaatgagcggcggcggacaccactgcggaaggcctcacaaccaggagggataggccacctctgggcatccttcagtggcaagctacgtcgcattgtagacagcaagtacttcaaccgaggcatcatggcagccatcctcgtcaatactctgagcatgggcgttgagtatcatgaacagcctgaggagctgaccaacgccctggagataagcaacatcgtgttcaccagcatgtttgccttggagatgctactgaagctgctggcctgcggcccactgggatacatccggaacccctacaacatcttcgatggcattgttgtcgtcataagtgtctgggagatcgtggggcaggcggacggtggcctgtctgtgctgcgcaccttcaggctgctgcgggtgctgaagctggtgcgcttcctgccggccctgcggcgccagctcgtggtgctcatgaggaccatggacaacgtggccaccttctgcatgctcctcatgctgttcatcttcatcttcagcatcctgggcatgcacctgttcggctgtaagttcagcctgaagacagactctggagacaccgtccctgacaggaagaacttcgactccctactgtgggccatcgtcaccgtgtttcagatcttgacacaggaagactggaacgtggttctgtacaacggcatggcctccacttcgtcctgggccgccctttactttgtggccctcatgacctttgggaactatgtgctcttcaacctgctggtagccatcctggtggaaggtttccaggcagagggtgacgccaccagatctgacaccgacgaggataagacgtctacccagctagagggagatttcgataagctcagagatcttcgagccacagagatgaagatgtattcactggcagtgacccctaacgggcacctagagggccgaggcagcctgccgccgcccctcatcactcacacggcagctacgcctatgcctactcccaaaagctccccaaacctggacgtggcccatgctctcctggactctcggcgcagcagcagcggctctgtggacccccagctgggggaccagaagtctctggccagcctccgcagctccccttgcaccccatggggccccaacagcgctgggagcagcaggcgctccagttggaacagcctgggccgcgcacccagcctcaaacgccgcagccagtgtggggagcgcgagtccctgctctctggagaggggaagggcagcaccgatgacgaggccgaggacagcagaccaagcacgggaacccacccaggggcctcgccagggccccgagccacgccactgcggcgtgccgagtcattggaccaccgcagcacgctggacctgtgtccaccacggcctgcggccctcctgccgaccaagttccatgactgcaacgggcagatggtggccctgcccagcgagttctttctgcgcatcgacagccacaaggaggatgcagcggagtttgatgatgacatagaggatagctgctgcttccgtctacacaaagtgctggaaccctatgcaccccagtggtgccgtagccgggagtcctgggccctgtatctcttcccaccgcagaacaggctacgcgtctcctgccagaaagtcatcgcacacaagatgtttgaccacgtggtccttgtcttcatcttcctcaactgtatcaccattgctctggagaggccagacattgacccaggcagcactgagcgggccttcctcagcgtctccaactacatcttcacagccatcttcgtggtggagatgatggtgaaggtggtagccctgggactgctgtggggtgaacatgcctacctacagagcagttggaatgtgctggacgggctgcttgtcctggtatccctggttgacatcatcgtggccatggcctcagctggcggtgccaagatcctaggcgtcctgcgtgtgctgcgcctgctgcggaccctgaggcctctgagggtcatcagccgagctccaggcctcaagctggttgtagagactctgatatcatcgctcaggcccattgggaacatcgtcctcatctgctgcgccttcttcatcatctttggcatcctcggggtgcagcttttcaagggcaaattctactactgcgagggcacagataccaggaatatcaccaccaaggccgagtgccatgctgcccactaccgctgggtgaggcgcaaatacaactttgacaacctgggtcaggcgctgatgtctctgttcgtgctgtcatctaaggatggctgggtaaacatcatgtatgacgggctggatgccgtgggcatcgaccagcagcccgtgcagaaccacaacccctggatgctgctctacttcatctccttcctgctcatcgtcagcttcttcgtgctcaacatgtttgtgggcgtggtggtggagaacttccacaagtgccggcagcaccaggaggctgaggaggctcggcgccgggaggagaaacggctgcggcgcctggagaggaggcgcaggaaggcccagcgccggccctactacgcagactattcacacactcgccgctccatccattcgctgtgcaccagccactacctggacctcttcatcaccttcatcatctgcctcaatgtcatcaccatgtccatggagcactacaaccagcccaagtccctggatgaggccctcaagtactgcaactacgtctttaccatcgtcttcgtctttgaggctgcactgaagctggtggcctttgggttccggaggttcttcaaggacaggtggaaccagctggacttggccatcgtcctcctatccatcatgggcattgcgctggaggagattgagatgaacgccgccctgcccatcaatcccaccatcatccgcatcatgcgtgtgcttcgaatcgcccgtgtgctgaagctactgaagatggccacaggcatgcgcgccttgctggatactgtggttcaagctctgcctcaggtagggaaccttggtcttcttttcatgctcctgttttttatctatgctgccctgggagtggagctgtttgggaggctagagtgcagcgaggataacccctgcgagggcctgagcaggcacgctaccttcaccaacttcggcatggccttcctcacactgtttcgagtgtccactggggacaactggaatgggattatgaaggataccctccgtgagtgtacccgtgaggacaagcactgcctcagctacctgcccgcgctctcacccgtctacttcgtcaccttcgtgctggtggctcagttcgtgctggtcaatgtggtggtggccgtgctcatgaagcacctggaggagagcaacaaggaggcccgcgaggatgcagagatggacgccgagatcgagctggagatggcacaggggtccacagcccagcccccacctacagcacaggaaagccaaggtacccagccagacaccccgaacctcctggtcgtgcgaaaagtatctgtgtccaggatgctctcgctgcccaatgacagctacatgttcaggccggtggctcccgcggctgccccacattcccacccactgcaggaagtggagatggagacctacacaggcccggtcacctctgctcactcgccacccctggagccccgcgcctctttccaggtcccatcagccgcgtcctccccagccagggtcagtgaccccctttgtgccctttcaccccggggtacaccccgctctctgagtctctcacggatactctgcagacaggaggccatgcactctgagtccctggaagggaaggttgatgatgttggaggagacagcatcccagactacacagagcctgctgaaaatatgtccacgagccaggcatcaacaggtgccccgaggtcccctccgtgctccccgcgacctgccagcgtccgtacccgcaagcacacgtttgggcaacgctgcatctccagccgccctcccaccctgggaggagatgaggctgaagcagcagacccagcagatgaggaggtcagccacatcaccagctcagcccacccctggccggctacagagccccacagccctgaggcctccccaacagcctctcctgtgaaaggcacaatgggcagtgggcgggacccacgcaggttctgcagtgtagatgctcagagcttcctggacaaaccaggtcggccagatgcacaacggtggtcctcagtggaactggataacggagaaagccacctagagtccggggaagtgaggggccgggcctcagagctcgaaccagctcttggggcacgaaggaagaagaagatgagccctccctgcatctccattgaacctcccactgaggatgagggctcttcccggccccctgcagccgaaggaggcaacactaccctgaggcgccgaaccccatcctgtgaggctgccctccatagggactgcccagagcctacagaaggcccaggcaccggaggggaccctgtagccaagggtgagcgctggggccaggcctcttgccgagcagagcatctgactgtccccaactttgcctttgagcctctggacatgggcggacctggtggagactgtttcttggacagtgaccaaagtgtgaccccagaacccagagtttcctctttgggggctatagtgcctctgatactagaaactgaactttctatgccctctggtgactgcccagagaaggaacaaggactgtacctcactgtgccccagacccccttgaagaaaccagggtctaccccagccactcctgccccagatgacagtggagatgagcctgtgtagatggggctgtgtgtccacagggctttggcattgaggttgttggctccctgcagggtggtagggccatgagtggaccctggcttaggccccactaaggcagagggaccgggagataaccatcccaggagaggcagcagacatcccgtctctgcaccatgacacaggagcagcctcgggccccacgagcctccctcgtggtgattcaggtttgggttttcctgagttttaaccaccaccagaagctgtaccaggaccaggtcatcagtctcaggaggagaggctgtgtcctgggaagga

SEQ ID NO: 2 - protein, 2359 aa, Rat
MTEGTLAADEVRVPLGASPPAPAAPVRASPASPGAPGREEQGGSGSGVLAPESPGTECGADLGADEEQPVPYPALAATVFFCLGQTTRPRSWCLRLVCNPWFEHISMLVIMLNCVTLGMFRPCEDVECRSERCSILEAFDDFIFAFFAVEMVIKMVALGLFGQKCYLGDTWNRLDFFIVMAGMMEYSLDGHNVSLSAIRTVRVLRPLRAINRVPSMRILVTLLLDTLPMLGNVLLLCFFVFFIFGIVGVQLWAGLLRNRCFLDSAFVRNNNLTFLRPYYQTEEGEENPFICSSRRDNGMQKCSHIPSRRELRVQCTLGWEAYGQPQAEDGGAGRNACINWNQYYNVCRSGEFNPHNGAINFDNIGYAWIAIFQVITLEGWVDIMYYVMDAHSFYNFIYFILLIIVGSFFMINLCLVVIATQFSETKQRENQLMREQRARYLSNDSTLASFSEPGSCYEELLKYVGHIFRKVKRRSLRLYARWQSRWRKKVDPSSTVHGQGPGRRPRRAGRRTASVHHLVYHHHHHHHHHYHFSHGGPRRPSPEPGAGDNRLVRACAPPSPPSPGHGPPDSESVHSIYHADCHVEGPQERARVAHSIATAASLKLASGLGTMNYPTILPSGTVNSKGGTSSRPKGLRGAGAPGAAVHSPLSLGSPRPYEKIQHVVGEQGLGRASSHLSGLSVPCPLPSPQAGTLTCELKSCPYCASALEDPEFEFSGSESGDSDAHGVYEFTQDVRHGDCRDPVQQPHEVGTPGHSNERRRTPLRKASQPGGIGHLWASFSGKLRRIVDSKYFNRGIMAAILVNTLSMGVEYHEQPEELTNALEISNIVFTSMFALEMLLKLLACGPLGYIRNPYNIFDGIVVVISVWEIVGQADGGLSVLRTFRLLRVLKLVRFLPALRRQLVVLMRTMDNVATFCMLLMLFIFIFSILGMHLFGCKFSLKTDSGDTVPDRKNFDSLLWAIVTVFQILTQEDWNVVLYNGMASTSSWAALYFVALMTFGNYVLFNLLVAILVEGFQAEGDATRSDTDEDKTSTQLEGDFDKLRDLRATEMKMYSLAVTPNGHLEGRGSLPPPLITHTAATPMPTPKSSPNLDVAHALLDSRRSSSGSVDPQLGDQKSLASLRSSPCTPWGPNSAGSSRRSSWNSLGRAPSLKRRSQCGERESLLSGEGKGSTDDEAEDSRPSTGTHPGASPGPRATPLRRAESLDHRSTLDLCPPRPAALLPTKFHDCNGQMVALPSEFFLRIDSHKEDAAEFDDDIEDSCCFRLHKVLEPYAPQWCRSRESWALYLFPPQNRLRVSCQKVIAHKMFDHVVLVFIFLNCITIALERPDIDPGSTERAFLSVSNYIFTAIFVVEMMVKVVALGLLWGEHAYLQSSWNVLDGLLVLVSLVDIIVAMASAGGAKILGVLRVLRLLRTLRPLRVISRAPGLKLVVETLISSLRPIGNIVLICCAFFIIFGILGVQLFKGKFYYCEGTDTRNITTKAECHAAHYRWVRRKYNFDNLGQALMSLFVLSSKDGWVNIMYDGLDAVGIDQQPVQNHNPWMLLYFISFLLIVSFFVLNMFVGVVVENFHKCRQHQEAEEARRREEKRLRRLERRRRKAQRRPYYADYSHTRRSIHSLCTSHYLDLFITFIICLNVITMSMEHYNQPKSLDEALKYCNYVFTIVFVFEAALKLVAFGFRRFFKDRWNQLDLAIVLLSIMGIALEEIEMNAALPINPTIIRIMRVLRIARVLKLLKMATGMRALLDTVVQALPQVGNLGLLFMLLFFIYAALGVELFGRLECSEDNPCEGLSRHATFTNFGMAFLTLFRVSTGDNWNGIMKDTLRECTREDKHCLSYLPALSPVYFVTFVLVAQFVLVNVVVAVLMKHLEESNKEAREDAEMDAEIELEMAQGSTAQPPPTAQESQGTQPDTPNLLVVRKVSVSRMLSLPNDSYMFRPVAPAAAPHSHPLQEVEMETYTGPVTSAHSPPLEPRASFQVPSAASSPARVSDPLCALSPRGTPRSLSLSRILCRQEAMHSESLEGKVDDVGGDSIPDYTEPAENMSTSQASTGAPRSPPCSPRPASVRTRKHTFGQRCISSRPPTLGGDEAEAADPADEEVSHITSSAHPWPATEPHSPEASPTASPVKGTMGSGRDPRRFCSVDAQSFLDKPGRPDAQRWSSVELDNGESHLESGEVRGRASELEPALGARRKKKMSPPCISIEPPTEDEGSSRPPAAEGGNTTLRRRTPSCEAALHRDCPEPTEGPGTGGDPVAKGERWGQASCRAEHLTVPNFAFEPLDMGGPGGDCFLDSDQSVTPEPRVSSLGAIVPLILETELSMPSGDCPEKEQGLYLTVPQTPLKKPGSTPATPAPDDSGDEPV

SEQ ID NO: 3 - DNA, 7432 bp, Rat
ccacggccacggggacgccgctagccaccggagcgaggtgctgccctccgccaccatgaccgagggcacgctggcagcggacgaagtccgggtgcccctgggcgcttcgccgccggcccctgcagcgccggtgagggcttccccagcgagccctggggcgccggggcgcgaggagcagggaggatccgggtcgggcgtgttggctcccgagagcccagggaccgagtgtggtgcggacctgggcgccgacgaggaacagccggtcccatacccagctctggctgccacagtcttcttctgcctcgggcaaaccacgcggccgcgcagctggtgcctccgactggtttgtaacccgtggttcgagcacatcagcatgctggtcatcatgctgaactgcgtgacactgggcatgttcaggccctgtgaggatgttgagtgccgctccgaacgttgcagcatcttggaggccttcgacgacttcatctttgccttcttcgccgtggagatggtgatcaagatggtggctttggggctgtttgggcaaaaatgctacctgggtgacacctggaacaggctggacttcttcattgtcatggcgggcatgatggagtactctctggacggacacaacgtgagcctctctgccatccgaaccgtgcgtgtgctgcggcccctccgcgccatcaaccgagtccccagtatgcggatcctggtcactctgctgctggacacgctgcccatgcttgggaatgtcctcctcctctgcttcttcgtcttcttcatcttcggcattgttggggtccagctctgggctggcctgcttcggaaccgatgcttcctggacagcgccttcgtcaggaacaacaacctgaccttcttgcggccatactaccagacggaggagggtgaggagaaccctttcatctgctcctcccgccgtgacaacggcatgcagaagtgctcgcacatccccagccgccgtgagcttcgagtgcagtgcacactcggctgggaggcctatgggcagccacaggctgaggatgggggtgctggccgcaacgcctgtatcaactggaaccagtattacaacgtgtgccgctcgggggaattcaaccctcacaacggtgccatcaacttcgacaacattggctacgcttggattgccatcttccaggtcatcacactggagggctgggtggacatcatgtactacgtcatggatgcccactcgttctacaacttcatctacttcatcctcctcatcattgtgggctccttcttcatgatcaacctgtgcctggtggtgatagccacacagttctcagagacaaagcaaagggaaaaccagctgatgcgagaacagcgggcccgctatctgtccaacgacagcactctggccagcttctcagagcccggcagctgctacgaggagctcctcaagtatgtaggccacatcttccggaaggttaaacgccgtagcctgcgtctttatgcccgctggcagagccgctggcgtaagaaggtggatcccagcagtaccgtgcatggccaaggccctgggcggcggccacgacgggcaggcaggcgtacagcttcagtgcaccatctggtctaccaccaccaccaccaccatcaccaccattaccactttagccacggtggcccacgcaggcccagcccagagccaggtgctggtgacaacaggttggtcagggcctgtgcgccaccctcgccgccatccccaggccatgggccaccagactctgagtctgtgcacagtatctaccatgctgactgccacgtggaggggccgcaggaacgagcccgagtggcacactccatagccactgctgctagcctcaagctggcctcaggtttgggtaccatgaactaccccaccatcctaccttcaggaacagtcaacagcaaaggtggcaccagctcacgacccaaggggctacgaggtgctggcgccccaggggctgcagtacacagccctctgagcctgggaagccccagaccctatgagaagatccagcatgtggtgggagaacaaggactaggccgagcctctagccacctgtcaggcctgagtgtgccttgccccctgcccagcccccaggctggcacgctgacctgtgagctgaagagctgcccatattgtgccagcgccctggaggaccccgagtttgaattcagtggctcagagagcggggactcggatgcccacggagtctatgagtttacccaggatgtacggcatggggattgtcgggaccctgtgcagcagccccatgaagtgggcacaccaggccacagcaatgagcggcggcggacaccactgcggaaggcctcacaaccaggagggataggccacctctgggcatccttcagtggcaagctacgtcgcattgtagacagcaagtacttcaaccgaggcatcatggcagccatcctcgtcaatactctgagcatgggcgttgagtatcatgaacagcctgaggagctgaccaacgccctggagataagcaacatcgtgttcaccagcatgtttgccttggagatgctactgaagctgctggcctgcggcccactgggatacatccggaacccctacaacatcttcgatggcattgttgtcgtcataagtgtctgggagatcgtggggcaggcggacggtggcctgtctgtgctgcgcaccttcaggctgctgcgggtgctgaagctggtgcgcttcctgccggccctgcggcgccagctcgtggtgctcatgaggaccatggacaacgtggccaccttctgcatgctcctcatgctgttcatcttcatcttcagcatcctgggcatgcacctgttcggctgtaagttcagcctgaagacagactctggagacaccgtccctgacaggaagaacttcgactccctactgtgggccatcgtcaccgtgtttcagatcttgacacaggaagactggaacgtggttctgtacaacggcatggcctccacttcgtcctgggccgccctttactttgtggccctcatgacctttgggaactatgtgctcttcaacctgctggtagccatcctggtggaaggtttccaggcagagggtgacgccaccagatctgacaccgacgaggataagacgtctacccagctagagggagatttcgataagctcagagatcttcgagccacagagatgaagatgtattcactggcagtgacccctaacgggcacctagagggccgaggcagcctgccgccgcccctcatcactcacacggcagctacgcctatgcctactcccaaaagctccccaaacctggacgtggcccatgctctcctggactctcggcgcagcagcagcggctctgtggacccccagctgggggaccagaagtctctggccagcctccgcagctccccttgcaccccatggggccccaacagcgctgggagcagcaggcgctccagttggaacagcctgggccgcgcacccagcctcaaacgccgcagccagtgtggggagcgcgagtccctgctctctggagaggggaagggcagcaccgatgacgaggccgaggacagcagaccaagcacgggaacccacccaggggcctcgccagggccccgagccacgccactgcggcgtgccgagtcattggaccaccgcagcacgctggacctgtgtccaccacggcctgcggccctcctgccgaccaagttccatgactgcaacgggcagatggtggccctgcccagcgagttctttctgcgcatcgacagccacaaggaggatgcagcggagtttgatgatgacatagaggatagctgctgcttccgtctacacaaagtgctggaaccctatgcaccccagtggtgccgtagccgggagtcctgggccctgtatctcttcccaccgcagaacaggctacgcgtctcctgccagaaagtcatcgcacacaagatgtttgaccacgtggtccttgtcttcatcttcctcaactgtatcaccattgctctggagaggccagacattgacccaggcagcactgagcgggccttcctcagcgtctccaactacatcttcacagccatcttcgtggtggagatgatggtgaaggtggtagccctgggactgctgtggggtgaacatgcctacctacagagcagttggaatgtgctggacgggctgcttgtcctggtatccctggttgacatcatcgtggccatggcctcagctggcggtgccaagatcctaggcgtcctgcgtgtgctgcgcctgctgcggaccctgaggcctctgagggtcatcagccgagctccaggcctcaagctggttgtagagactctgatatcatcgctcaggcccattgggaacatcgtcctcatctgctgcgccttcttcatcatctttggcatcctcggggtgcagcttttcaagggcaaattctactactgcgagggcacagataccaggaatatcaccaccaaggccgagtgccatgctgcccactaccgctgggtgaggcgcaaatacaactttgacaacctgggtcaggcgctgatgtctctgttcgtgctgtcatctaaggatggctgggtaaacatcatgtatgacgggctggatgccgtgggcatcgaccagcagcccgtgcagaaccacaacccctggatgctgctctacttcatctccttcctgctcatcgtcagcttcttcgtgctcaacatgtttgtgggcgtggtggtggagaacttccacaagtgccggcagcaccaggaggctgaggaggctcggcgccgggaggagaaacggctgcggcgcctggagaggaggcgcaggaaggcccagcgccggccctactacgcagactattcacacactcgccgctccatccattcgctgtgcaccagccactacctggacctcttcatcaccttcatcatctgcctcaatgtcatcaccatgtccatggagcactacaaccagcccaagtccctggatgaggccctcaagtactgcaactacgtctttaccatcgtcttcgtctttgaggctgcactgaagctggtggcctttgggttccggaggttcttcaaggacaggtggaaccagctggacttggccatcgtcctcctatccatcatgggcattgcgctggaggagattgagatgaacgccgccctgcccatcaatcccaccatcatccgcatcatgcgtgtgcttcgaatcgcccgtgtgctgaagctactgaagatggccacaggcatgcgcgccttgctggatactgtggttcaagctctgcctcaggtagggaaccttggtcttcttttcatgctcctgttttttatctatgctgccctgggagtggagctgtttgggaggctagagtgcagcgaggataacccctgcgagggcctgagcaggcacgctaccttcaccaacttcggcatggccttcctcacactgtttcgagtgtccactggggacaactggaatgggattatgaaggataccctccgtgagtgtacccgtgaggacaagcactgcctcagctacctgcccgcgctctcacccgtctacttcgtcaccttcgtgctggtggctcagttcgtgctggtcaatgtggtggtggccgtgctcatgaagcacctggaggagagcaacaaggaggcccgcgaggatgcagagatggacgccgagatcgagctggagatggcacaggggtccacagcccagcccccacctacagcacaggaaagccaaggtacccagccagacaccccgaacctcctggtcgtgcgaaaagtatctgtgtccaggatgctctcgctgcccaatgacagctacatgttcaggccggtggctcccgcggctgccccacattcccacccactgcaggaagtggagatggagacctacacaggcccggtcacctctgctcactcgccacccctggagccccgcgcctctttccaggtcccatcagccgcgtcctccccagccagggtcagtgaccccctttgtgccctttcaccccggggtacaccccgctctctgagtctctcacggatactctgcagacaggaggccatgcactctgagtccctggaagggaaggttgatgatgttggaggagacagcatcccagactacacagagcctgctgaaaatatgtccacgagccaggcatcaacaggtgccccgaggtcccctccgtgctccccgcgacctgccagcgtccgtacccgcaagcacacgtttgggcaacgctgcatctccagccgccctcccaccctgggaggagatgaggctgaagcagcagacccagcagatgaggaggtcagccacatcaccagctcagcccacccctggccggctacagagccccacagccctgaggcctccccaacagcctctcctgtgaaaggcacaatgggcagtgggcgggacccacgcaggttctgcagtgtagatgctcagagcttcctggacaaaccaggtcggccagatgcacaacggtggtcctcagtggaactggataacggagaaagccacctagagtccggggaagtgaggggccgggcctcagagctcgaaccagctcttggggcacgaaggaagaagaagatgagccctccctgcatctccattgaacctcccactgaggatgagggctcttcccggccccctgcagccgaaggaggcaacactaccctgaggcgccgaaccccatcctgtgaggctgccctccatagggactgcccagagcctacagaaggcccaggcaccggaggggaccctgtagccaagggtgagcgctggggccaggcctcttgccgagcagagcatctgactgtccccaactttgcctttgagcctctggacatgggcggacctggtggagactgtttcttggacagtgaccaaagtgtgaccccagaacccagagtttcctctttgggggctatagtgcctctgatactagaaactgaactttctatgccctctggtgactgcccagagaaggaacaaggactgtacctcactgtgccccagacccccttgaagaaaccagggtctaccccagccactcctgccccagatgacagtggagatgagcctgtgtagatggggctgtgtgtccacagggctttggcattgaggttgttggctccctgcagggtggtagggccatgagtggaccctggcttaggccccactaaggcagagggaccgggagataaccatcccaggagaggcagcagacatcccgtctctgcaccatgacacaggagcagcctcgggccccacgagcctccctcgtggtgattcaggtttgggttttcctgagttttaaccaccaccagaagctgtaccaggaccaggtcatcagtctcaggaggagaggctgtgtcctgggaagga

SEQ ID NO: 4 - protein, 2359 aa, Rat; identical to SEQ ID NO: 2

SEQ ID NO: 5 - DNA, 7277 bp, Rat
ccacggggacgccgctagccaccggagcgaggtgctgccctccgccaccatgaccgagggcacgctggcagcggacgaagtccgggtgcccctgggcgcttcgccgccggcccctgcagcgccggtgagggcttccccagcgagccctggggcgccggggcgcgaggagcagggaggatccgggtcgggcgtgttggctcccgagagcccagggaccgagtgtggtgcggacctgggcgccgacgaggaacagccggtcccatacccagctctggctgccacagtcttcttctgcctcgggcaaaccacgcggccgcgcagctggtgcctccgactggtttgtaacccgtggttcgagcacatcagcatgctggtcatcatgctgaactgcgtgacactgggcatgttcaggccctgtgaggatgttgagtgccgctccgaacgttgcagcatcttggaggccttcgacgacttcatctttgccttcttcgccgtggagatggtgatcaagatggtggctttggggctgtttgggcaaaaatgctacctgggtgacacctggaacaggctggacttcttcattgtcatggcgggcatgatggagtactctctggacggacacaacgtgagcctctctgccatccgaaccgtgcgtgtgctgcggcccctccgcgccatcaaccgagtccccagtatgcggatcctggtcactctgctgctggacacgctgcccatgcttgggaatgtcctcctcctctgcttcttcgtcttcttcatcttcggcattgttggggtccagctctgggctggcctgcttcggaaccgatgcttcctggacagcgccttcgtcaggaacaacaacctgaccttcttgcggccatactaccagacggaggagggtgaggagaaccctttcatctgctcctcccgccgtgacaacggcatgcagaagtgctcgcacatccccagccgccgtgagcttcgagtgcagtgcacactcggctgggaggcctatgggcagccacaggctgaggatgggggtgctggccgcaacgcctgtatcaactggaaccagtattacaacgtgtgccgctcgggggaattcaaccctcacaacggtgccatcaacttcgacaacattggctacgcttggattgccatcttccaggtcatcacactggagggctgggtggacatcatgtactacgtcatggatgcccactcgttctacaacttcatctacttcatcctcctcatcattgtgggctccttcttcatgatcaacctgtgcctggtggtgatagccacacagttctcagagacaaagcaaagggaaaaccagctgatgcgagaacagcgggcccgctatctgtccaacgacagcactctggccagcttctcagagcccggcagctgctacgaggagctcctcaagtatgtaggccacatcttccggaaggttaaacgccgtagcctgcgtctttatgcccgctggcagagccgctggcgtaagaaggtggatcccagcagtaccgtgcatggccaaggccctgggcggcggccacgacgggcaggcaggcgtacagcttcagtgcaccatctggtctaccaccaccaccaccaccatcaccaccattaccactttagccacggtggcccacgcaggcccagcccagagccaggtgctggtgacaacaggttggtcagggcctgtgcgccaccctcgccgccatccccaggccatgggccaccagactctgagtctgtgcacagtatctaccatgctgactgccacgtggaggggccgcaggaacgagcccgagtggcacactccatagccactgctgctagcctcaagctggcctcaggtttgggtaccatgaactaccccaccatcctaccttcaggaacagtcaacagcaaaggtggcaccagctcacgacccaaggggctacgaggtgctggcgccccaggggctgcagtacacagccctctgagcctgggaagccccagaccctatgagaagatccagcatgtggtgggagaacaaggactaggccgagcctctagccacctgtcaggcctgagtgtgccttgccccctgcccagcccccaggctggcacgctgacctgtgagctgaagagctgcccatattgtgccagcgccctggaggaccccgagtttgaattcagtggctcagagagcggggactcggatgcccacggagtctatgagtttacccaggatgtacggcatggggattgtcgggaccctgtgcagcagccccatgaagtgggcacaccaggccacagcaatgagcggcggcggacaccactgcggaaggcctcacaaccaggagggataggccacctctgggcatccttcagtggcaagctacgtcgcattgtagacagcaagtacttcaaccgaggcatcatggcagccatcctcgtcaatactctgagcatgggcgttgagtatcatgaacagcctgaggagctgaccaacgccctggagataagcaacatcgtgttcaccagcatgtttgccttggagatgctactgaagctgctggcctgcggcccactgggatacatccggaacccctacaacatcttcgatggcattgttgtcgtcataagtgtctgggagatcgtggggcaggcagacggtggcctgtctgtgctgcgcaccttcaggctgctgcgggtgctgaagctggtgcgcttcctgccggccctgcggcgccagctcgtggtgctcatgaggaccatggacaacgtggccaccttctgcatgctcctcatgctgttcatcttcatcttcagcatcctgggcatgcacctgttcggctgtaagttcagcctgaagacagactctggagacaccgtccctgacaggaagaacttcgactccctactgtgggccatcgtcaccgtgtttcagatcttgacacaggaagactggaacgtggttctgtacaacggcatggcctccacttcgtcctgggccgccctttactttgtggccctcatgacctttgggaactatgtgctcttcaacctgctggtagccatcctggtggaaggtttccaggcagagggtgacgccaccagatctgacaccgacgaggataagacgtctacccagctagagggagatttcgataagctcagagatcttcgagccacagagatgaagatgtattcactggcagtgacccctaacgggcacctagagggccgaggcagcctgccgccgcccctcatcactcacacggcagctacgcctatgcctactcccaaaagctccccaaacctggacgtggcccatgctctcctggactctcggcgcagcagcagcggctctgtggacccccagctgggggaccagaagtctctggccagcctccgcagctccccttgcaccccatggggccccaacagcgctgggagcagcaggcgctccagttggaacagcctgggccgcgcacccagcctcaaacgccgcagccagtgtggggagcgcgagtccctgctctctggagaggggaagggcagcaccgatgacgaggccgaggacagcagaccaagcacgggaacccacccaggggcctcgccagggccccgagccacgccactgcggcgtgccgagtcattggaccaccgcagcacgctggacctgtgtccaccacggcctgcggccctcctgccgaccaagttccatgactgcaacgggcagatggtggccctgcccagcgagttctttctgcgcatcgacagccacaaggaggatgcagcggagtttgatgatgacatagaggatagctgctgcttccgtctacacaaagtgctggaaccctatgcaccccagtggtgccgtagccgggagtcctgggccctgtatctcttcccaccgcagaacaggctacgcgtctcctgccagaaagtcatcgcacacaagatgtttgaccacgtggtccttgtcttcatcttcctcaactgtatcaccattgctctggagaggccagacattgacccaggcagcactgagcgggccttcctcagcgtctccaactacatcttcacagccatcttcgtggtggagatgatggtgaaggtggtagccctgggactgctgtggggtgaacatgcctacctacagagcagttggaatgtgctggacgggctgcttgtcctggtatccctggttgacatcatcgtggccatggcctcagctggcggtgccaagatcctaggcgtcctgcgtgtgctgcgcctgctgcggaccctgaggcctctgagggtcatcagccgagctccaggcctcaagctggttgtagagactctgatatcatcgctcaggcccattgggaacatcgtcctcatctgctgcgccttcttcatcatctttggcatcctcggggtgcagcttttcaagggcaaattctactactgcgagggcacagataccaggaatatcaccaccaaggccgagtgccatgctgcccactaccgctgggtgaggcgcaaatacaactttgacaacctgggtcaggcgctgatgtctctgttcgtgctgtcatctaaggatggctgggtaaacatcatgtatgacgggctggatgccgtgggcatcgaccagcagcccgtgcagaaccacaacccctggatgctgctctacttcatctccttcctgctcatcgtcagcttcttcgtgctcaacatgtttgtgggcgtggtggtggagaacttccacaagtgccggcagcaccaggaggctgaggaggctcggcgccgggaggagaaacggctgcggcgcctggagaggaggcgcaggaaggcccagcgccggccctactacgcagactattcacacactcgccgctccatccattcgctgtgcaccagccactacctggacctcttcatcaccttcatcatctgcctcaatgtcatcaccatgtccatggagcactacaaccagcccaagtccctggatgaggccctcaagtactgcaactacgtctttaccatcgtcttcgtctttgaggctgcactgaagctggtggcctttgggttccggaggttcttcaaggacaggtggaaccagctggacttggccatcgtcctcctatccatcatgggcattgcgctggaggagattgagatgaacgccgccctgcccatcaatcccaccatcatccgcatcatgcgtgtgcttcgaatcgcccgtgtgctgaagctactgaagatggccacaggcatgcgcgccttgctggatactgtggttcaagctctgcctcaggtagggaaccttggtcttcttttcatgctcctgttttttatctatgctgccctgggagtggagctgtttgggaggctagagtgcagcgaggataacccctgcgagggcctgagcaggcacgctaccttcaccaacttcggcatggccttcctcacactgttccgagtgtccactggggacaactggaatgggattatgaaggataccctccgtgagtgtacccgtgaggacaagcactgcctcagctacctgcccgcgctctcacccgtctacttcgtcaccttcgtgctggtggctcagttcgtgctggtcaatgtggtggtggccgtgctcatgaagcacctggaggagagcaacaaggaggcccgcgaggatgcagagatggacgccgagatcgagctggagatggcacaggggtccacagcccagcccccacctacagcacaggaaagccaaggtacccagccagacaccccgaacctcctggtcgtgcgaaaagtatctgtgtccaggatgctctcgctgcccaatgacagctacatgttcaggccggtggctcccgcggctgccccacattcccacccactgcaggaagtggagatggagacctacacaggcccggtcacctctgctcactcgccacccctggagccccgcgcctctttccaggtcccatcagccgcgtcctccccagccagggtcagtgaccccctttgtgccctttcaccccggggtacaccccgctctctgagtctctcacggatactctgcagacaggaggccatgcactctgagtccctggaagggaaggttgatgatgttggaggagacagcatcccagactacacagagcctgctgaaaatatgtccacgagccaggcatcaacaggtgccccgaggtcccctccgtgctccccgcgacctgccagcgtccgtacccgcaagcacacgtttgggcaacgctgcatctccagccgccctcccaccctgggaggagatgaggctgaagcagcagacccagcagatgaggaggtcagccacatcaccagctcagcccacccctggccggctacagagccccacagccctgaggcctccccaacagcctctcctgtgaaaggcacaatgggcagtgggcgggacccacgcaggttctgcagtgtagatgctcagagcttcctggacaaaccaggtcggccagatgcacaacggtggtcctcagtggaactggataacggagaaagccacctagagtccggggaagtgaggggccgggcctcagagctcgaaccagctcttgggtcacgaaggaagaagaagatgagccctccctgcatctccattgaacctcccactgaggatgagggctcttcccggccccctgcagccgaaggaggcaacactaccctgaggcgccgaaccccatcctgtgaggctgccctccatagggactgcccagagcctacagaaggcccaggcaccggaggggaccctgtagccaagggtgagcgctggggccaggcctcttgccgagcagagcatctgactgtccccaactttgcctttgagcctctggacatgggcggacctggtggagactgtttcttggacagtgaccaaagtgtgaccccagaacccagagtttcctctttgggggctatagtgcctctgatactagaaactgaactttctatgccctctggtgactgcccagagaaggaacaaggactgtacctcactgtgccccagacccccttgaagaaaccagggtctaccccagccactcctgccccagatgacagtggagatgagcctgtgtagatggggctgcgtgtccacagggctttggcattgaggttgttggctccctgcagggtggtagggccatgagtggaccctggcttaggccccactaaggcagagggaccgggagataaccatcccaggagaggcagcagacatcccgtct

SEQ ID NO: 6 - protein, 2359 aa, Rat
MTEGTLAADEVRVPLGASPPAPAAPVRASPASPGAPGREEQGGSGSGVLAPESPGTECGADLGADEEQPVPYPALAATVFFCLGQTTRPRSWCLRLVCNPWFEHISMLVIMLNCVTLGMFRPCEDVECRSERCSILEAFDDFIFAFFAVEMVIKMVALGLFGQKCYLGDTWNRLDFFIVMAGMMEYSLDGHNVSLSAIRTVRVLRPLRAINRVPSMRILVTLLLDTLPMLGNVLLLCFFVFFIFGIVGVQLWAGLLRNRCFLDSAFVRNNNLTFLRPYYQTEEGEENPFICSSRRDNGMQKCSHIPSRRELRVQCTLGWEAYGQPQAEDGGAGRNACINWNQYYNVCRSGEFNPHNGAINFDNIGYAWIAIFQVITLEGWVDIMYYVMDAHSFYNFIYFILLIIVGSFFMINLCLVVIATQFSETKQRENQLMREQRARYLSNDSTLASFSEPGSCYEELLKYVGHIFRKVKRRSLRLYARWQSRWRKKVDPSSTVHGQGPGRRPRRAGRRTASVHHLVYHHHHHHHHHYHFSHGGPRRPSPEPGAGDNRLVRACAPPSPPSPGHGPPDSESVHSIYHADCHVEGPQERARVAHSIATAASLKLASGLGTMNYPTILPSGTVNSKGGTSSRPKGLRGAGAPGAAVHSPLSLGSPRPYEKIQHVVGEQGLGRASSHLSGLSVPCPLPSPQAGTLTCELKSCPYCASALEDPEFEFSGSESGDSDAHGVYEFTQDVRHGDCRDPVQQPHEVGTPGHSNERRRTPLRKASQPGGIGHLWASFSGKLRRIVDSKYFNRGIMAAILVNTLSMGVEYHEQPEELTNALEISNIVFTSMFALEMLLKLLACGPLGYIRNPYNIFDGIVVVISVWEIVGQADGGLSVLRTFRLLRVLKLVRFLPALRRQLVVLMRTMDNVATFCMLLMLFIFIFSILGMHLFGCKFSLKTDSGDTVPDRKNFDSLLWAIVTVFQILTQEDWNVVLYNGMASTSSWAALYFVALMTFGNYVLFNLLVAILVEGFQAEGDATRSDTDEDKTSTQLEGDFDKLRDLRATEMKMYSLAVTPNGHLEGRGSLPPPLITHTAATPMPTPKSSPNLDVAHALLDSRRSSSGSVDPQLGDQKSLASLRSSPCTPWGPNSAGSSRRSSWNSLGRAPSLKRRSQCGERESLLSGEGKGSTDDEAEDSRPSTGTHPGASPGPRATPLRRAESLDHRSTLDLCPPRPAALLPTKFHDCNGQMVALPSEFFLRIDSHKEDAAEFDDDIEDSCCFRLHKVLEPYAPQWCRSRESWALYLFPPQNRLRVSCQKVIAHKMFDHVVLVFIFLNCITIALERPDIDPGSTERAFLSVSNYIFTAIFVVEMMVKVVALGLLWGEHAYLQSSWNVLDGLLVLVSLVDIIVAMASAGGAKILGVLRVLRLLRTLRPLRVISRAPGLKLVVETLISSLRPIGNIVLICCAFFIIFGILGVQLFKGKFYYCEGTDTRNITTKAECHAAHYRWVRRKYNFDNLGQALMSLFVLSSKDGWVNIMYDGLDAVGIDQQPVQNHNPWMLLYFISFLLIVSFFVLNMFVGVVVENFHKCRQHQEAEEARRREEKRLRRLERRRRKAQRRPYYADYSHTRRSIHSLCTSHYLDLFITFIICLNVITMSMEHYNQPKSLDEALKYCNYVFTIVFVFEAALKLVAFGFRRFFKDRWNQLDLAIVLLSIMGIALEEIEMNAALPINPTIIRIMRVLRIARVLKLLKMATGMRALLDTVVQALPQVGNLGLLFMLLFFIYAALGVELFGRLECSEDNPCEGLSRHATFTNFGMAFLTLFRVSTGDNWNGIMKDTLRECTREDKHCLSYLPALSPVYFVTFVLVAQFVLVNVVVAVLMKHLEESNKEAREDAEMDAEIELEMAQGSTAQPPPTAQESQGTQPDTPNLLVVRKVSVSRMLSLPNDSYMFRPVAPAAAPHSHPLQEVEMETYTGPVTSAHSPPLEPRASFQVPSAASSPARVSDPLCALSPRGTPRSLSLSRILCRQEAMHSESLEGKVDDVGGDSIPDYTEPAENMSTSQASTGAPRSPPCSPRPASVRTRKHTFGQRCISSRPPTLGGDEAEAADPADEEVSHITSSAHPWPATEPHSPEASPTASPVKGTMGSGRDPRRFCSVDAQSFLDKPGRPDAQRWSSVELDNGESHLESGEVRGRASELEPALGSRRKKKMSPPCISIEPPTEDEGSSRPPAAEGGNTTLRRRTPSCEAALHRDCPEPTEGPGTGGDPVAKGERWGQASCRAEHLTVPNFAFEPLDMGGPGGDCFLDSDQSVTPEPRVSSLGAIVPLILETELSMPSGDCPEKEQGLYLTVPQTPLKKPGSTPATPAPDDSGDEPV